(19) United States
(12) Patent Application Publication
(10) Pub. No.: US 2026/0315431 A1
(43) Pub. Date: Oct. 15, 2026

(54) NANOMATERIAL ENCAPSULATED BIOFILM INHIBITORS FOR DENTAL CARIES TREATMENT

(71) Applicant: THE UAB RESEARCH FOUNDATION, BIRMINGHAM, AL (US)

(72) Inventors: Sadanandan E. Velu, BIRMINGHAM, AL (US); Eugenia Kharlampieva, BIRMINGHAM, AL (US); Parmanand Ahiwar, BIRMINGHAM, AL (US); Veronika Kozlovskaya, BIRMINGHAM, AL (US)

(21) Appl. No.: 19/482,984

(22) PCT Filed: May 21, 2024
(86) PCT No.: PCT/US2024/030331

§ 371 Date: Nov. 10, 2025

Related U.S. Application Data
(60) Provisional application No. 63/503,801, filed on May 23, 2023.
(60) Provisional application No. 63/584,657, filed on Sep. 22, 2023.

(51) Int. Cl. A61K 31/343; A61K 9/00; A61K 9/51; A61P 1/02

(52) U.S. Cl. CPC A61K 31/343; A61K 9/0053; A61K 9/5138; A61K 9/5146; A61P 1/02

(57) ABSTRACT

The present disclosure provides for compositions including aurone-based small molecules encapsulated into hydrogel particles. The disclosure also provides for pharmaceutical compositions including the hydrogel encapsulated aurones and methods of use of the hydrogel encapsulated aurones. Compounds and pharmaceutical compositions of the present disclosure can be used in combination with one or more other therapeutic agents for treating biofilms, such as Streptococcus mutans (S. mutans) biofilm, dental caries, and other diseases.

### CROSS-REFERENCE TO RELATED APPLICATIONS

This application claims the benefit of U.S. Provisional Application entitled “NANOMATERIAL ENCAPSULATED BIOFILM INHIBITORS FOR DENTAL CARIES TREATMENT” and having Ser. No. 63/503,801, filed May 23, 2023, and this application claims the benefit of U.S. Provisional Application entitled “NANOMATERIAL ENCAPSULATED BIOFILM INHIBITORS FOR DENTAL CARIES TREATMENT” and having Ser. No. 63/584,657, filed Sep. 22, 2023, each of which is herein incorporated by reference in their entireties.

### STATEMENT ON FUNDING PROVIDED BY THE U.S. GOVERNMENT

This invention was made with Government support under contract 1R21DE028349 awarded by the National Institutes of Health. The Government has certain rights in the invention.

### BACKGROUND

Dental caries, commonly known as tooth decay, is a ubiquitous bacterial infectious disease that causes demineralization of enamel and dentin. Although dental plaque contains more than 700 bacterial species living in complex bacterial communities called biofilms, the gram-positive bacterium Streptococcus mutans, characterized by its ability to form tenacious biofilms is considered to be the primary etiological agent for this disease.

Current antimicrobial treatments for dental caries such as oral rinses affect both pathogenic and commensal bacteria alike. Therefore, it would be beneficial to develop new caries treatments that do not have adverse impact on the growth of oral commensal species.

### SUMMARY

Aspects of the present disclosure provide aurone-based small molecules encapsulated into hydrogel particles. The disclosure also provides for pharmaceutical compositions including the hydrogel encapsulated aurones and methods of use of the hydrogel encapsulated aurones.

The present disclosure provides for a composition comprising a plurality of hydrogel particles and a plurality of small molecules, wherein the small molecules are encapsulated inside the hydrogel particles and wherein the small molecules have the following structure:

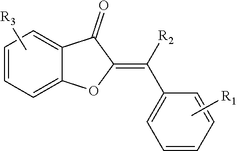

wherein each R₁ is independently selected from hydrogen, a substituted or unsubstituted C1-C3 alkyl group, a substituted or unsubstituted C1-C3 alkoxyl group, and OH, wherein R2 is hydrogen, a substituted or unsubstituted C1-C3 alkyl group, a substituted or unsubstituted C1-C3 alkoxyl group, and OH, wherein each R₃ is independently selected from hydrogen, a substituted or unsubstituted C1-C3 alkyl group, a substituted or unsubstituted C1-C3 alkoxyl group, and OH.

The present disclosure provides for a pharmaceutical composition comprising a therapeutically effective amount of the composition of any one of claims 1-16 to treat a condition.

The present disclosure provides for a method for treating dental caries comprising contacting a biofilm or contacting a cell capable of forming a biofilm with a therapeutically effective amount of the composition or pharmaceutical composition of any one of claims 1-20.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1.1 illustrates the identification of two potent lead compounds G43 and IIIC5.

FIG. 1.2A illustrates methoxy and hydroxy aurones.

FIG. 1.2B illustrates Scheme 1, which is a synthesis of substituted aurones.

FIGS. 1.3A-C illustrate planktonic growth inhibitory activities of chalcones (3a-f), methoxyaurones (MA1-6), and hydroxyaurones (HA2-6). FIG. 1.3A illustrates a graph showing that S. mutans UA159 were co-incubated with 50 μM of chalcones 3a-f and the planktonic growth was measured at OD₄₇₀. FIG. 1.3B illustrates a graph showing that S. mutans UA159 were co-incubated with 50 μM of methoxy aurones, MA1-6 and the planktonic growth was measured at OD₄₇₀. FIG. 1.3C illustrates that S. mutans UA159 were co-incubated with 50 μM of hydroxyaurones, HA2-6 and the planktonic growth was measured at OD₄₇₀. Each experiment was repeated three times with triplicate microwells for each compound. Statistical significance was tested with one-way ANOVA. p<0.0001.

FIGS. 1.4A-C illustrate biofilm inhibitory activities of chalcones (3a-f), methoxyaurones (MA1-6) and hydroxyaurones (HA2-6). FIG. 1.4A illustrates that S. mutans UA159 were co-incubated with 50 μM of chalcones 3a-f and biofilm formation was measured at OD₅₆₂ using the crystal violet protocol. FIG. 1.4B illustrates that S. mutans UA159 were co-incubated with 50 μM of methoxyaurones, MA1-6 and biofilm formation was measured at OD₅₆₂ using the crystal violet protocol. FIG. 1.4 illustrates that S. mutans UA159 were co-incubated with 50 μM of hydroxyaurones HA2-6 and biofilm formation was measured at OD₅₆₂ using the crystal violet protocol. Each experiment was repeated three times with triplicate microwells for each compound. Statistical significance was tested with one-way ANOVA. p<0.0001.

FIGS. 1.5A-B illustrate inhibitory activities of hydroxyaurones (HA2-6) against commensal biofilms. FIG. 1.5A illustrates that S. gordonii DL1 were co-incubated with 50 mM of hydroxyaurones, HA2-6 or G43 and biofilm formation was measured at OD₅₆₂ using the crystal violet protocol. FIG. 1.5B illustrates S. sanguinis SK36 were co-incubated with 50 mM of hydroxyaurones, HA2-6 or G43 and biofilm formation was measured at OD₅₆₂ using the crystal violet protocol. Each experiment was repeated three times with triplicate microwells for each compound. Statistical significance was tested with one-way ANOVA. p<0.0001.

FIGS. 1.6A-E illustrate the biofilm inhibitory activities of compound HA5. FIG. 1.6A illustrates that S. mutans UA159 were co-incubated with HA5 at various concentrations and biofilm formation was measured at OD₅₆₂ using the crystal violet protocol. FIG. 1.6B illustrates Gtfs precipitated from S. mutans culture were co-incubated with HA5 at various concentrations and the glucan production was quantified using cascade blue staining and subsequent image processing with ImageJ. FIG. 1.6C illustrates representative fluorescence microscopy images of UA159 biofilms after 16 h of treatment with various concentrations of HA5. Bacterial cells were stained with Syto-9 (green, panel I); glucans were stained with Cascade Blue-dextran conjugated dye (blue, panel II); eDNA was stained with propidium iodide (red, panel III) and a merged image of all three staining images (panel IV). FIG. 1.6D illustrates S. mutans UA159, S. gordonii DL1 and S. sanguinis SK36 were co-incubated with HA5 at 50 μM and 100 μM and their growth were measured at OD₄₇₀. FIG. 1.6E illustrates the chemical structure of HA5. Each of the biofilm, glucan and growth assays were conducted in triplicate and statistical significance was tested with one-way ANOVA. p<0.0001.

FIG. 1.7A illustrates optical images of empty (PMAA)₅ hydrogels microparticles. FIG. 1.7B illustrates HA5-loaded hydrogel HEBI and HA5 in methanol (insert B). FIG. 1.7C illustrates Atomic Force Microscopy (AFM) topography images of a tooth surface with height of 280 nm. D) AFM image after (PMAA)₅ hydrogel adsorption, cubical hydrogel particles are clearly seen sticking to the tooth surface. FIG. 1.7E illustrates amplitude error image of empty (PMAA)₅ hydrogels dried on the surface of a tooth. Scan size is 20 μm2 in both images, the height (z)-scale is 1.7 μm. FIG. 1.7F illustrates S. mutans UA159 and two bacterial commensal species S. gordonii DL1 or S. sanguinis SK36 were co-incubated with HA5 or HEBI at 25 μM and their growth was measured at OD₄₇₀. FIG. 1.7G illustrates S. mutans UA159, S. gordonii DL1 or S. sanguinis SK36 were co-incubated with 25 mM of HA5 or HEBI and biofilm formation was measured at OD₅₆₂ using the crystal violet protocol. Each of the biofilm and growth assays were conducted in triplicate and statistical significance was tested with one-way ANOVA. p<0.0001.

FIG. 2.1 illustrates inhibitors of S. mutans glucosyl transferases and biofilm. FIG. 2.2A-E illustrates biofilm and growth inhibitory activities of the compound HA6. FIG. 2.2A illustrates that S. mutans UA159 were co-incubated with HA6 at various concentrations and biofilm formation was measured at OD₅₆₂ using the crystal violet protocol and IC₅₀ value was determined. FIG. 2.2B illustrates that S. mutans UA159, S. gordonii DL1 and S. sanguinis SK36 were co-incubated with HA6 at 25 μM and 50 μM and their planktonic growth were measured at OD₄₇₀. FIG. 2.2C illustrates that S. gordonii DL1 were co-incubated with HA6 at 25 μM and biofilm formation was measured at OD₅₆₂ using the crystal violet protocol. FIG. 2.2D illustrates that S. sanguinis SK36 were co-incubated with HA6 at 25 μM and biofilm formation was measured at OD₅₆₂ using the crystal violet protocol. FIG. 2.2E illustrates representative fluorescence microscopy images of UA159 biofilms after 16 h of treatment with various concentrations of HA6. Bacterial cells were stained with Syto-9 (green, panel-I); glucans were stained with Cascade Blue dextran conjugated dye (blue, panel-II); eDNA was stained with propidium iodide (red, panel-III), and a merged image of all three staining images (panel-IV).

FIG. 2.3A-D illustrate compounds HA5 and HA6 do not perturb oral microbiome significantly. Oral microbiome samples were obtained from individual rats at the following time points: before the experiment (Native), after inoculation of S. mutans and the start of a caries-promoting diet (Sm+CPD), after two weeks of treatment with the compounds (2-week), and at the end of the study (END). The microbiota between groups at different time points were analyzed for diversity and composition. FIG. 2.3A illustrates Phyla composition in all groups. Each color represents 1 phylum, and the length of the bar reflects relative abundance. The major phyla detected throughout the study were Firmicutes, Proteobacteria, Bacteroides, Actinobacteria, Verrucomicrobia, Epsilonbacteraeota, Tenericutes, Cyanobacteria, and Spirochaetes. n=5 per group. FIG. 2.3B illustrates Family-level composition within phylum Firmicutes. The major families detected within the phylum are Lachnospiraceae, Ruminococcaceae, Lactobacillus, Erysipelotrichaceae, and Streptococcaceae, n=5. FIG. 2.3C illustrates alpha diversity of the oral bacterial community structure at the genus level of each treatment group. Three diversity indices: Chao-1, Shannon, and Simpson were calculated, before (Native) and after (END) the treatment interventions and compared against the untreated group. n=5. FIG. 2.3D illustrates beta diversity (Bray-Curtis index) of the oral bacterial community structure at the genus level for each treatment group is represented by the principal coordinates analysis (PCoA) plots where the samples were clustered (ellipses) based on the time points as depicted in the color legend: native (red), Sm+CPD (green), 2-week (blue) and END (purple). Each dot represents 1 rat, n=5.

FIG. 2.4A illustrates the synthesis of PVPON₈-PDMS₆₄-PVPON₈ triblock copolymer by RAFT copolymerization. FIG. 2.4B illustrates 1H-NMR spectrum of PVPON₈-PDMS₆₄-PVPON₈ triblock copolymer. FIG. 2.4C illustrates photographs of the polymersome solution at pH=7.4 and after its exposure to pH=3 after 24 h. FIG. 2.4D illustrates optical density of the PVPON-b-PDMS-b-PVPON polymersomes before and after 24-h exposure to pH=3 solution at 37° C., FIG. 2.4E illustrates hydrodynamic size (diameter, nm) of empty polymersome and PEHA5 and PEHA6 as measured by DLS. FIG. 2.4F illustrates UV-visible spectra of HA5 and HA6 (solid lines) and PEHA5 and PEHA6 (dotted lines) in water.

FIG. 2.5A-E illustrates biofilm and growth inhibitory activities of PEHA5. FIG. 2.5A illustrates that S. mutans UA159 were co-incubated with various concentrations of PEHA5, and biofilm formation was measured at OD₅₆₂ using the crystal violet protocol. FIG. 2.5B illustrates that S. mutans UA159 were co-incubated with empty polymersome vesicles, 50 μM of HA5 or PEHA5 and the biofilm formation compared to control (1% DMSO) was measured at OD₅₆₂ using the crystal violet protocol. FIG. 2.5C illustrates that S. mutans UA159 were co-incubated with various concentrations of PEHA5, and the planktonic growth was measured at OD₄₇₀. FIG. 2.5D illustrates that S. mutans UA159 and two bacterial commensal species S. sanguinis SK36 and S. gordonii DL1 co-incubated with PEHA5 or HA5 at 50 μM and their planktonic growth was measured at OD₄₇₀. FIG. 2.5E illustrates representative fluorescence microscopy images of UA159 biofilms after 16 h of treatment with various concentrations of PEHA5. Bacterial cells were stained with Syto-9 (green, panel-I); glucans were stained with Cascade Blue-dextran conjugated dye (blue, panel-II); eDNA was stained with propidium iodide (red, panel-III), and a merged image of all three staining images (panel-IV).

FIG. 2.6A-E illustrates that biofilm and growth inhibitory activities of PEHA6. FIG. 2.6A illustrates that S. mutans UA159 were co-incubated with various concentrations of PEHA6, and biofilm formation was measured at OD₅₆₂ using the crystal violet protocol. FIG. 2.6B illustrates that S. mutans UA159 were co-incubated with 50 μM of HA5, HA6, or PEHA6 and the biofilm formation compared to control (1% DMSO) was measured at OD₅₆₂ using the crystal violet protocol. FIG. 2.6C illustrates that S. mutans UA159 were co-incubated with various concentrations of PEHA6, and planktonic growth was measured at OD₄₇₀. FIG. 2.6D illustrates S. mutans UA159 co-incubated with HA5, HA6 or PEHA6 at 50 UM and their planktonic growth was measured at OD₄₇₀. FIG. 2.6E illustrates representative fluorescence microscopy images of UA159 biofilms after 16 h of treatment with various concentrations of PEHA6. Bacterial cells were stained with Syto-9 (green, panel-l); glucans were stained with Cascade Blue-dextran conjugated dye (blue, panel-II); eDNA was stained with propidium iodide (red, panel-III), and a merged image of all three staining images (panel-IV).

### DETAILED DESCRIPTION

The present disclosure provides for compositions including aurone-based small molecules encapsulated into hydrogel particles. The disclosure also provides for pharmaceutical compositions including the hydrogel encapsulated aurones and methods of use of the hydrogel encapsulated aurones.

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

It will be understood by those skilled in the art that the moieties substituted can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “acyl” as used herein, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfonyl (e.g. allylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like. The term “acyl” as used herein refers to a group —C(O)R₂₆, where R₂₆ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

The terms “administering” and “administration” as used herein refer to introducing a composition (e.g., a vaccine, adjuvant, or immunogenic composition) of the present disclosure into a subject. As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. A preferred route of administration of the vaccine composition is intravenous.

The terms “alkoxyl” or “alkoxyalkyl” as used herein refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, t-butoxyl, and pentoxyl.

The term “alkyl”, either alone or within other terms such as “thioalkyl” and “arylalkyl”, as used herein, means a monovalent, saturated hydrocarbon radical which may be a straight chain (i.e. linear) or a branched chain. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group comprising methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkenyl” as used herein is a hydrocarbon group of from 2 to 24 carbon atoms or 2 to 8 carbon atoms or 2 to 6 carbon atoms with a structural formula containing at least one carbon-carbon double bond. Asymmetric structures such as (R₁R₂)C═C(R₃R₄) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

As used herein, “alkynyl” or “alkynyl group” refers to straight or branched chain hydrocarbon groups having 2 to 40, 2 to 20, 2 to 10, or 2 to 5 carbon atoms and at least one triple carbon to carbon bond, such as ethynyl. Reference to “alkynyl” or “alkynyl group” includes unsubstituted and substituted forms of the hydrocarbon moiety.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The Ar (e.g., Ar₁, Ar₂, etc) group is an aromatic system or group such as an aryl group. “Aryl”, as used herein, refers to C5-C20-membered aromatic, heterocyclic, fused aromatic, fused heterocyclic, biaromatic, or bihetereocyclic ring systems. In an aspect, “aryl”, can include 5-, 6-, 7-, 8-, 9-, and 10-membered single-ring aromatic groups that may include from zero to four heteroatoms, for example, functional groups that correspond to benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems (C₅-C₃₀) having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

In some aspects, a structure of a compound can be represented by a formula:

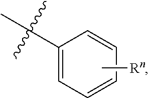

which is understood to be equivalent to a formula:

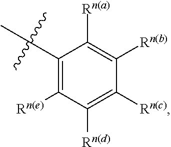

wherein n is typically an integer. That is, Rn is understood to represent five independent substituents, Rn(a), Rn(b), Rn(c), Rn(d), and Rn(e). By “independent substituents,” it is meant that each R substituent can be independently defined. For example, if in one instance Rn(a) is halogen, then Rn(b) is not necessarily halogen in that instance.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert. alkoxycarbonyl such as tert-butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert·butylcarborlyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltert·butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like. A therapeutic composition of the disclosure may comprise a carrier, such as one or more of a polymer, carbohydrate, peptide or derivative thereof, which may be directly or indirectly covalently attached to the compound. A carrier may be substituted with substituents described herein including without limitation one or more alkyl, amino, nitro, halogen, thiol, thioalkyl, sulfate, sulfonyl, sulfinyl, sulfoxide, hydroxyl groups. In aspects of the disclosure the carrier is an amino acid including alanine, glycine, praline, methionine, serine, threonine, asparagine, alanyl-alanyl, prolyl-methionyl, or glycyl-glycyl. A carrier can also include a molecule that targets a compound of the disclosure to a particular tissue or organ.

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21.sup.st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbitol, and the like. For oral administration in a liquid form, the chug components may be combined with any oral, non-toxic, pharmaceutically, acceptable inert carrier such as ethanol, glycerol, water, and the like. Suitable binders (e.g., gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use. Pharmaceutically acceptable carriers may also include a dentifrice.

The term “dentifrice” or “dentifrice composition” refers to products used for purposes of administering therapeutic agent(s) to the oral cavity of a subject, during which time they are retained in the oral cavity for a time sufficient to allow for contact with substantially all surfaces of the teeth and/or oral tissues. A dentifrice composition may be in the form of a paste, powder, liquid, mouthwash, mouth rinse, chewing gum, tablet, cream, dental strips, gels, flosses, and the like.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Additionally, the term “pharmaceutically acceptable” describes a material that is not biologically or otherwise undesirable, i.e., without causing an unacceptable level of undesirable biological effects or interacting in a deleterious manner.

As used herein, the terms “effective amount” and “amount effective” refer to an amount that is sufficient to achieve the desired result or to have an effect on an undesired condition or prevention of a disease or condition (e.g., biofilms, dental caries) or enhance and/or tune the immune system of the subject to the desirable responses (e.g., to Streptococcus mutans (S. mutans)). For example, a “therapeutically effective amount” refers to an amount that is sufficient to achieve the desired therapeutic result or to have an effect on undesired symptoms or prevention of a disease or condition (e.g., biofilms, dental caries) and/or tune the immune system of the subject to the desirable responses but is generally insufficient to cause adverse side effects. The specific therapeutically effective dose level for any particular patient will depend upon a variety of factors including; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof (e.g., biofilms, dental caries), such as infections and consequences thereof and/or tuning the immune system of the subject to the desirable responses (e.g., to Streptococcus mutans (S. mutans)). The effect can be therapeutic in terms of a partial or complete cure of a disease, condition, symptom or adverse effect attributed to the disease, disorder, or condition. The term “treatment” as used herein can include any treatment of infections in a subject, particularly a human and can include any one or more of the following: (a) preventing the disease from occurring in a subject which may be predisposed to the disease or infection but has not yet been diagnosed as having it; (b) inhibiting the disease or infection, i.e., arresting its development; and (c) relieving the disease or infection i.e., mitigating or ameliorating the disease and/or its symptoms or conditions, (d) and/or tune the immune system of the subject to the desirable responses (e.g., to Streptococcus mutans (S. mutans)). The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition, and/or tuning the immune system of the subject to the desirable responses (e.g., to Streptococcus mutans (S. mutans)). Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition (e.g., biofilms, dental caries), or side effect, or to decreasing in the rate of advancement of a disease, disorder, condition, or side effect and/or tuning the immune system of the subject to the desirable responses (e.g., to Streptococcus mutans (S. mutans)).

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

### Discussion

The present disclosure provides for compositions including aurone-based small molecules encapsulated into hydrogel particles. The disclosure also provides for pharmaceutical compositions including the hydrogel encapsulated aurones and methods of use of the hydrogel encapsulated aurones. Compounds and pharmaceutical compositions of the present disclosure can be used in combination with one or more other therapeutic agents for treating biofilms, such as Streptococcus mutans (S. mutans) biofilm, dental caries, and other diseases. For example, compounds and pharmaceutical compositions of the present disclosure can be used to selectively target S. mutans biofilm and S. mutans biofilm formation without affecting the growth of oral commensal bacteria.

In one aspect, the aurone-based small molecules can have the following structure:

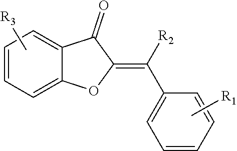

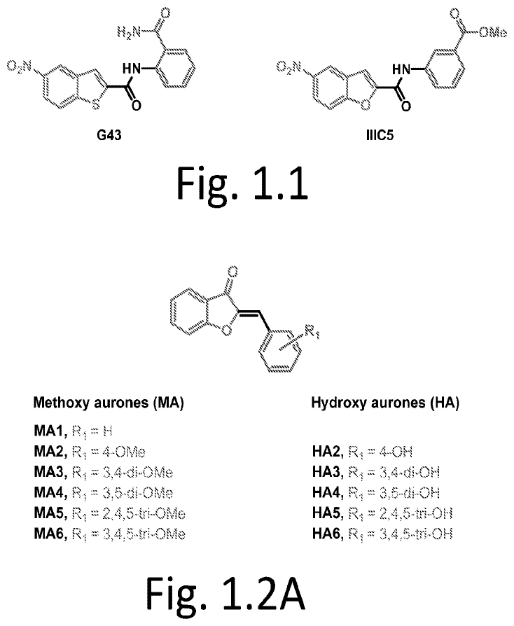

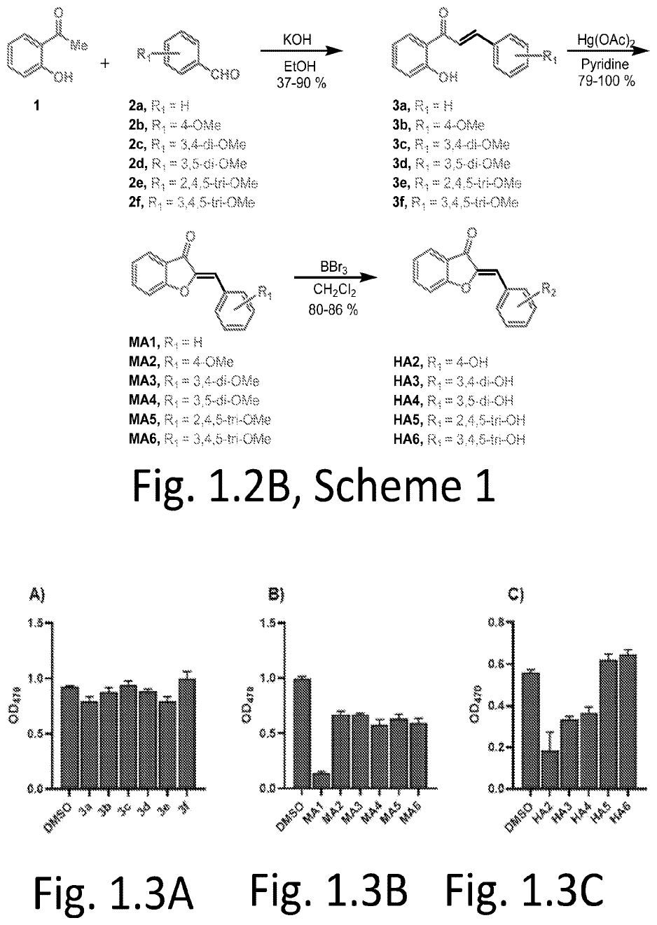

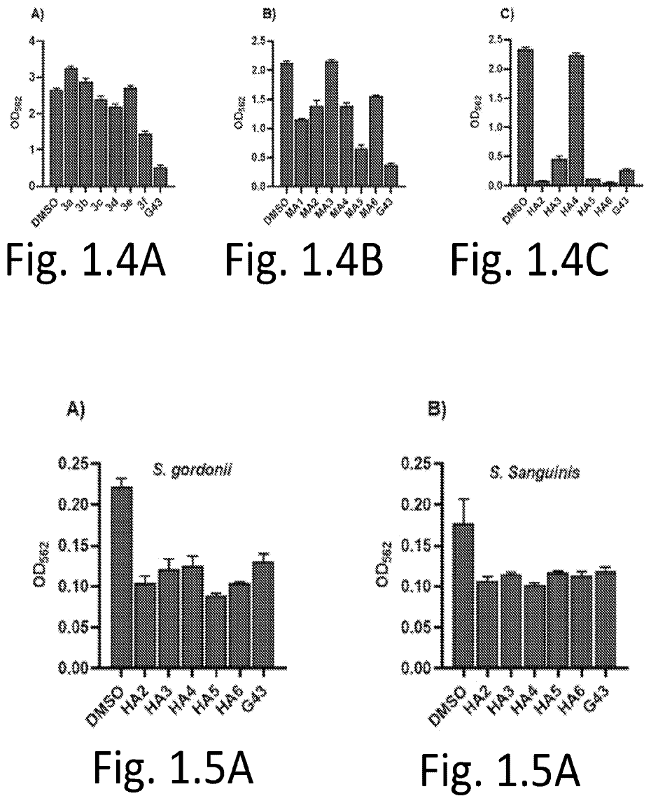

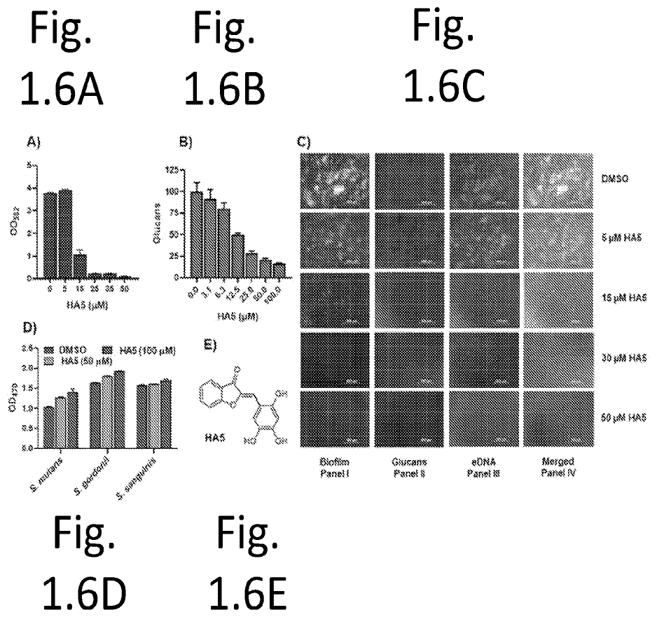

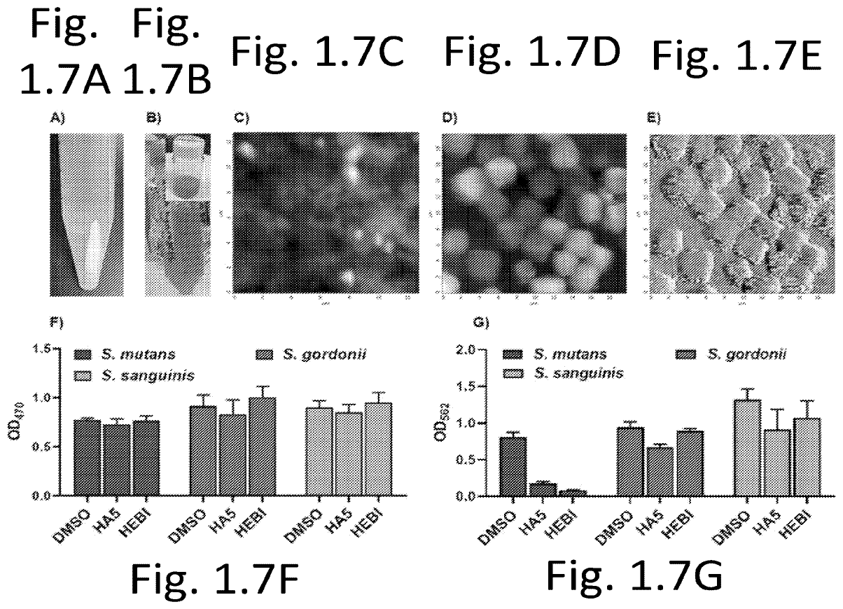

1 to 5 R₁ groups can be present in the structure above, and each R₁ can independently be hydrogen, a substituted or unsubstituted C1-C3 alkyl group, a substituted or unsubstituted C1-C3 alkoxyl group, or OH. In another aspect, each R₁ can independently be hydrogen, methoxy, or OH. In an aspect, 1, 2, or 3 R₁ groups can be present. For example, R₁ can be H or OH, or the structure can include two R₁ groups each being —OMe or OH, or the structure can include three R₁ groups each being —OMe or OH. R2 can be hydrogen, a substituted or unsubstituted C1-C3 alkyl group, a substituted or unsubstituted C1-C3 alkoxyl group, or OH. 1 to 5 R₃ groups can be present in the structure above, and each R₃ can independently be hydrogen, a substituted or unsubstituted C1-C3 alkyl group, a substituted or unsubstituted C1-C3 alkoxyl group, or OH. In another aspect, each R₃ can independently be hydrogen, methoxy, or OH. In an aspect, 1, 2, or 3 R₃ groups can be present. Illustrative compounds are shown in FIGS. 1.2A, 1.2B, and 2.1. Methods of forming these compounds are provided for in Example 1 and 2.

In an aspect, the hydrogel particles can include multiple layers of a polymer or co-polymer, where a plurality of aurone-based small molecules can be within the polymer or co-polymer. In another aspect, the polymer or co-polymer can form a vesicle that can include a plurality of aurone-based small molecules disposed within the vesical and/or within the polymer or co-polymer.

In an aspect, the hydrogel particles can be comprised of multiple layers of a cross-linked biocompatible polymer. The number of polymer layers can range 3 to 7, 4 to 7, 4 to 6, or 5 to 7. In some embodiments, the number of polymer layers is 5. The hydrogel can further comprise pH-sensitive cross-linkers including, but not limited to, ethylene diamine, cystamine, dithiobis(succinimidyl propionate), adipic acid dihydrazide, or any combination thereof. In some aspects, the polymer can be a carboxylic acid-based polymer, for example poly(methacrylic acid) (PMAA). The weight of the cross-linked PMAA can range from about 10 kDa to 400 kDa, about 20 to 300 kDa, about 30 to 200 kDa, about 40 to 150 kDa, or about 50 to 100 kDa. The hydrogel particle size (e.g., diameter) can be precisely selected. For example, the hydrogel particles can have diameter of about 1 to 10 μm, about 3 to 8 μm, about 4 to 6 μm, about 2 to 5 μm, or about 5 to 9 μm. More specifically, the particle diameter can be about 1 μm, 2 μm, 3 μm, 4 μm, 5 μm, 6 μm, 7 μm, 8 μm, 9 μm, or 10 μm. In some aspects, the hydrogel particles can be cubic in shape. The loading capacity of the hydrogel particles can range from about 0.003 to about 0.008 ng of aurone-based small molecule per particle or about 0.004 to about 0.007 ng of aurone-based small molecule per particle. Additional details are provided in Example 1.

In an aspect, the hydrogel particle can be a vesical that includes the aurone-based small molecules within the vesical and/or on the polymer or co-polymer that forms the vesicle. The vesical can include polymer or co-polymers of poly(N-vinylpyrrolidone) (PVPON) and poly(dimethylsiloxane) (PDMS). The weight of the polymer or co-polymer can range from about 10 kDa to 400 kDa, about 20 to 300 kDa, about 30 to 200 kDa, about 40 to 150 kDa, or about 50 to 100 kDa. The hydrogel particles size (e.g., diameter) can be precisely selected. For example, the hydrogel particles can have diameter of about 20 to 50 nm, about 25 to 45 nm, or about 30 nm. In an aspect, the hydrogen particle can be made of pH-responsive block copolymer vesicles to generate polymersome-encapsulated biofilm inhibitors. In an aspect, the polymersome is made from a poly(N-vinylpyrrolidone)₈-block-poly(dimethylsiloxane)₆₄-block-poly(N-vinyl-pyrrolidone)₈(PVPON₈-PDMS₆₄-PVPON₈) triblock copolymer. Additional details are provided in Example 2.

The pharmaceutical compositions provided for in this disclosure can include a therapeutically effective amount of hydrogel encapsulated aurones to treat a condition (e.g., dental caries) in a subject (e.g., animal or human subject). In some aspects, the pharmaceutical composition also includes a pharmaceutically acceptable carrier. The pharmaceutically acceptable carrier can be a dentifrice. The present disclosure also provides for methods of treating dental caries, biofilms, and the like by contacting a biofilm or a cell capable of forming a biofilm with a therapeutically effective amount of the hydrogel encapsulated aurone composition or pharmaceutical composition. In some aspects, the cell capable of forming the biofilm is S. mutans.

### Pharmaceutical Formulations and Routes of Administration

Embodiments of the present disclosure include the agent (e.g., hydrogel encapsulated aurones) as identified herein and can be formulated with one or more pharmaceutically acceptable excipients, diluents, carriers and/or adjuvants. In addition, embodiments of the present disclosure include the agent formulated with one or more pharmaceutically acceptable auxiliary substances. In particular the agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the agent can be administered to the subject using any means capable of resulting in the desired effect. Thus, the agent can be incorporated into a variety of formulations for therapeutic administration. For example, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent can be utilized in aerosol formulation to be administered via inhalation. Embodiments of the agent can be formulated into pressurized acceptable propellants such as dichlorodifluoromethane, propane, nitrogen and the like.

Furthermore, embodiments of the agent can be made into suppositories by mixing with a variety of bases such as emulsifying bases or water-soluble bases. Embodiments of the agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the agent in a composition as a solution in sterile water, normal saline or another pharmaceutically acceptable carrier.

Embodiments of the agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent can be accomplished using any of a variety of refillable, pump systems. Pumps provide consistent, controlled release over time. In some embodiments, the agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the agent can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho) esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the agent (e.g., hydrogel encapsulated aurones) can be administered to a subject in one or more doses. Those of skill will readily appreciate that dose levels can vary as a function of the specific the agent administered, the severity of the symptoms and the susceptibility of the subject to side effects. Preferred dosages for a given compound are readily determinable by those of skill in the art by a variety of means.

In an embodiment, multiple doses of the agent are administered. The frequency of administration of the agent can vary depending on any of a variety of factors, e.g., severity of the symptoms, and the like. For example, in an embodiment, the agent can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, theagent is administered continuously.

The duration of administration of the agent, e.g., the period of time over the agent is administered, can vary, depending on any of a variety of factors, e.g., patient response, etc. For example, the agent in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

Dosage at concentrations as high as 60 micrograms/kilograms that are non-toxic. Also lower concentrations, such as 1~4 micrograms/kilogram, show biological activity in in vivo systems. The concentration in in vitro established at 10⁻⁹-10⁻⁶ M are active and this concentration is expected to be achieved in the cell environment. (See Slominski A T, Janjetovic Z, Fuller B E, Zmijewski M A, Tuckey R C, et al. (2010) Products of vitamin D3 or 7-dehydrocholesterol metabolism by cytochrome P450scc show anti-leukemia effects, having low or absent calcemic activity. PLoS ONE 5(3): e990; Slominski A T, Kim T-K., Janjetovic Z, Tuckey R C, Bieniek, R, Yue Y, Li W, Chen J, Miller D, Chen T, Holick M (2011) 20-hydroxyvitamin D2 is a non-calcemic analog of vitamin D with potent antiproliferative and prodifferentiation activities in normal and malignant cells. Am J Physiol: Cell Physiol 300:C526-C541; Wang J, Slominski A T, Tuckey R C, Janjetovic Z, Kulkarni A, Chen J, Postlethwaite A, Miller D, Li W (2012) 20-Hydroxylvitamin D3 possesses high efficacy against proliferation of cancer cells while being non-toxic. Anticancer Res 32:739-746; Slominski A, Janjetovic Z, Tuckey R C, Nguyen M N, Bhattacharya K G, Wang J, Li W, Jiao Y, Gu W, Brown M, Postlethwaite A E (2013) 20-hydroxyvitamin D3, noncalcemic product of CYP11A1 action on vitamin D3, exhibits potent antifibrogenic activity in vivo. J Clin Endocrinol Metab 98, E298-E30; Chen, J., J. Wang, T. Kim, E. Tieu, E. Tamg, Lin Z, D. Kovacic, D. Miller, A. Postlethwaite, R. Tuckey, A. Slominski and W. Li (2014). Novel Vitamin D Analogs as Potential Therapeutics: The Metabolism, Toxicity Profiling, and Antiproliferative Activity. Anticancer Res 34:2153-2163.)

In an aspect, the dosage for administering to a subject (e.g., a mammal such as a human) having a condition (e.g., COVID-19) of any single agent the present disclosure is about 2 to 60 micrograms/kilogram or a combination of agents, each agent can be about 2 to 60 micrograms/kilogram.

### Routes of Administration

Embodiments of the present disclosure provide methods and compositions for the administration of the agent (e.g., hydrogel encapsulated aurones) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An agent can be administered in a single dose or in multiple doses.

Embodiments of the agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the agent. Where systemic delivery is desired, administration typically involves invasive or systemically absorbed topical or mucosal administration of pharmaceutical preparations.

In an embodiment, the agent can also be delivered to the subject by enteral administration. Enteral routes of administration include, but are not limited to, oral and rectal (e.g., using a suppository) delivery.

Methods of administration of the agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Most tooth and gum related diseases are associated with bacterial infections. Among these, dental caries (tooth decay) is a ubiquitous disease that affects much of the human population. Dental caries is a multifactorial disease that causes localized destruction of susceptible dental tissues¹. Dental caries is identified as the most prevalent disease worldwide in a recent Lancet study of global burden of 328 major diseases². Despite its general classification as a ‘life-style-related’ disease, dental caries poses a significant challenge as it results in tooth loss, infection, and in some cases, even death by sepsis^{3, 4}. Current treatments for this disease have severe limitations. The conventional oral hygiene practices such as brushing or mouthwashes are not highly effective due to the rapid re-colonization of the bacteria⁵. Fluoride sealants and varnishes are commonly used to prevent dental caries in children⁶. While there is a general consensus on the safety of fluoride treatments⁷, their high fluoride content (1-5%) and potential neurotoxic effects are a concern⁸. The antimicrobial agents used in mouthwashes such as chlorhexidine, xylitol, silver diamine fluoride and delmopinol lack selectivity, affecting both pathogenic and commensal beneficial species alike giving rise to undesired side effects such as vomiting, diarrhea, addiction, or teeth discoloration⁹. In addition, the biofilm nature of cariogenic bacteria makes it resistant to traditional anti-microbial treatments¹⁰. A few preventive and therapeutic strategies are under investigation by targeting different virulent determinants of S. mutans¹¹. However, small molecules derived from natural products that possess antibacterial activities and antibiofilm properties are randomly identified inhibitors that lack selectivity towards pathogenic biofilms and the in vivo applications of these inhibitors are unclear¹².

Dental plaque comprises more than 700 bacterial species living in complex communities called biofilms⁴. It is initiated by the attachment of commensal streptococci such as Streptococcus sanguinis and Streptococcus gordonii to the saliva-coated tooth surface, which then engage in developing intra- and inter-species bacterial interactions¹³ Under disease conditions, the delicate balance between commensal and pathogenic members of the plaque bacteria is disturbed, leading to an overgrowth of pathogenic species¹⁴. Streptococcus mutans has been implicated as the major etiological agent in the initiation and propagation of this disease¹⁵. The formation of tenacious biofilms is the hallmark of S. mutans induced cariogenesis. Therefore, the studies aimed at developing dental caries treatments should focus on identifying selective inhibitors of biofilms that do not affect the growth of oral commensal bacteria.

Major virulence factors of S. mutans that significantly contribute to its ability to form cariogenic biofilm are its extracellular glucosyl transferases (Gtfs)¹⁶. Most strains of S. mutans harbor three distinct gtf genes expressing different Gtf activities. The genes gtfB and gtfD produce GtfB and GtfD enzymes respectively and synthesize predominantly water-insoluble and soluble glucans¹⁷ correspondingly, while gtfC, encodes for GtfC, an enzyme that synthesizes both water-insoluble and soluble glucans¹⁸. S. mutans GtfB and GtfC are essential for glucan synthesis, bacterial colonization and cariogenesis. Therefore, small molecules inhibitors of S. mutans Gtfs^{19, 20, 21} have potential application in treating and preventing dental caries.

Many anti-biofilm agents display poor efficacy within the oral cavity due to poor solubility, inability to penetrate biofilms and lack of ability to retain in the locally infected areas. Given these challenges, antibacterial nanoparticles have generated recent interest due to their potential applications in anti-caries research. Examples of these are silver nanoparticles in the prevention of dental caries²², farnesol and myricetin co-loaded nanoparticles to inhibit biofilms²³, pH responsive materials to deliver farnesol²⁴, porous silicon microparticles to mitigate cariogenic biofilm²⁵, ferumoxytol nanoparticles²⁶ poly(ethylenimine)²⁷ and chitosan nanoparticles²⁸ with strong antibacterial activity against S. mutans. Several nano systems for controlled release of anti-caries drugs have also been explored including mesoporous silica nanoparticle²⁹, liposome³⁰, halloysite nano-tube³¹ polyamidoamine³² and dextran-coated Iron oxide nanoparticles (nanozymes)³³. Despite the flurry of these recent studies, none of these agents are translated for clinical use as their in vivo efficacies are either modest or not proven.

Under physiological conditions, the human salivary system maintains a healthy pH range of 6.0-7.5 in the oral cavity³⁴ using three buffer systems: 1) bicarbonate, 2) phosphate, and 3) salivary proteins³⁵. A salivary pH below 5.5 is potentially harmful to the hard and soft tissues in the oral cavity³⁶. Under pathogenic oral conditions, biofilms ferment dietary carbohydrates to produce acidic byproducts such as lactic acid, which decreases the pH and causes the demineralization of tooth enamel³⁷. Therefore, a drug that can specifically inhibit the biofilm delivered into the oral cavity in a pH-responsive manner would be highly desirable. Since the pH level in the oral cavity is critical for the demineralization of tooth enamel, our efforts were focused on developing a novel drug delivery system with built-in pH-sensitivity for the delivery of biofilm inhibitors as an anti-caries treatment.

Our recent studies aimed at developing selective small molecule inhibitors of S. mutans biofilm targeted the S. mutans' surface enzymes, Gtfs^{19, 20, 21}. These studies have resulted in the identification of two potent lead compounds G43 and IIIC5 (FIG. 1.1)¹⁹. The goals of present study were to improve the solubility of the lead biofilm inhibitor IIIC5, to encapsulate the optimized lead in pH-responsive hydrogel microparticles and to explore its biofilm and growth inhibitory activities in vitro and anti-virulence activities in vivo.

### Results and Discussion

Design and synthesis of biofilm inhibiting aurone compounds. Our initial efforts to prepare pH-responsive hydrogel encapsulated biofilm inhibitors using compounds G43 or IIIC5 did not yield the expected results due to the low solubility (10-25 μg/mL) of these inhibitors. Therefore, efforts were made to modify the structure of IIIC5 to improve solubility. Specifically, analogs of IIIC5 were prepared by substituting the benzofuran ring with a structurally similar aurone ring, removing the nitro, amide, and ester groups, and by introducing multiple hydrophilic OMe or OH groups on the phenyl ring. This study resulted in the identification of several aurone derivatives (MA1-6 and HA2-6, FIG. 1.2), of which the hydroxyaurones were found to have the desired solubility required for the hydrogel encapsulation while maintaining the potency and selectivity of biofilm inhibition.

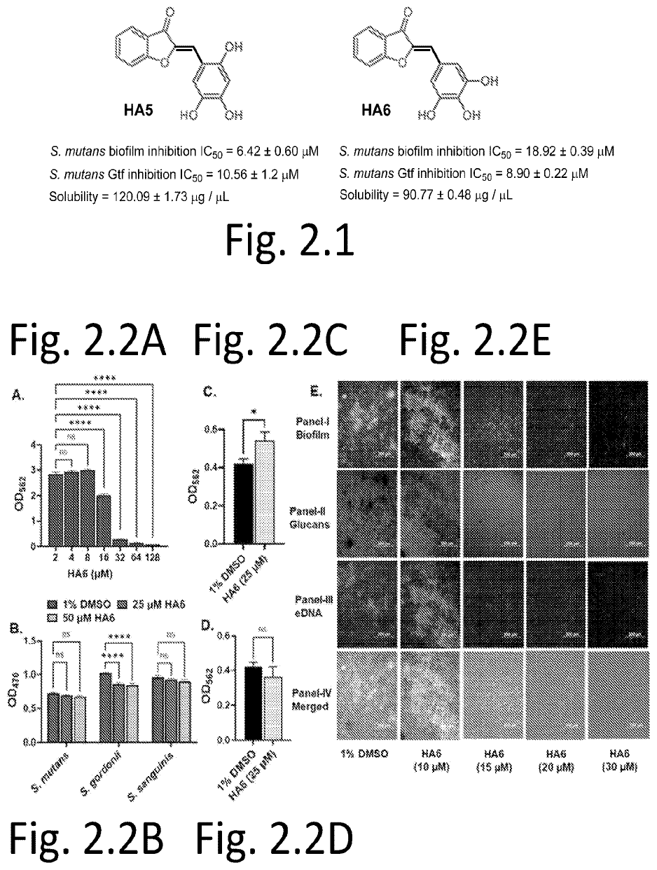

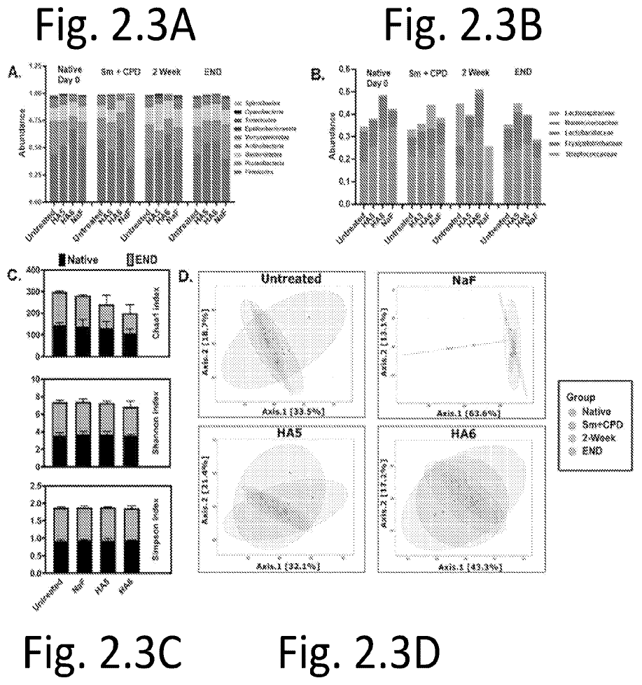

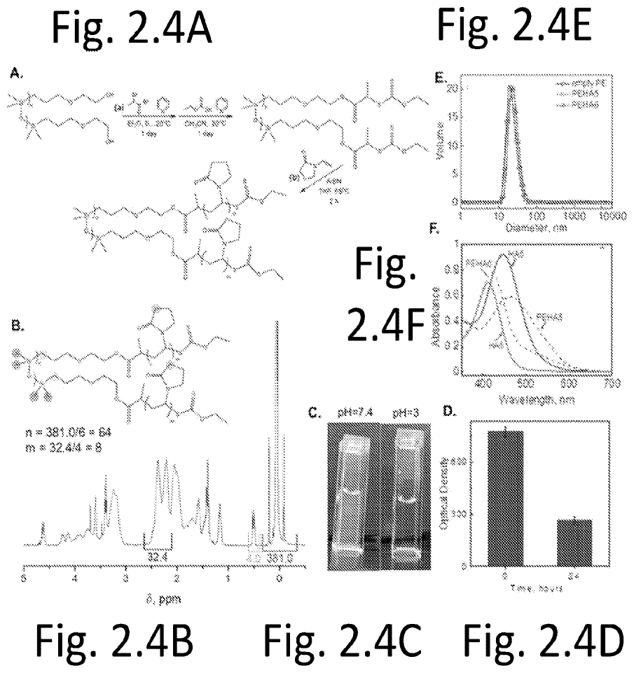

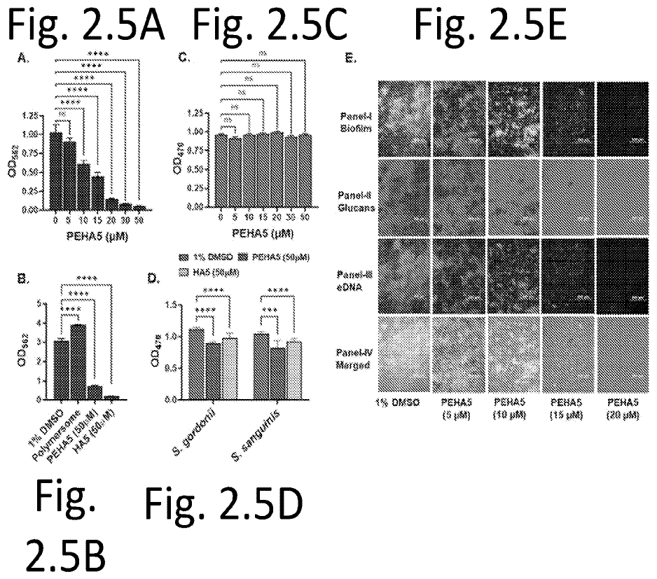

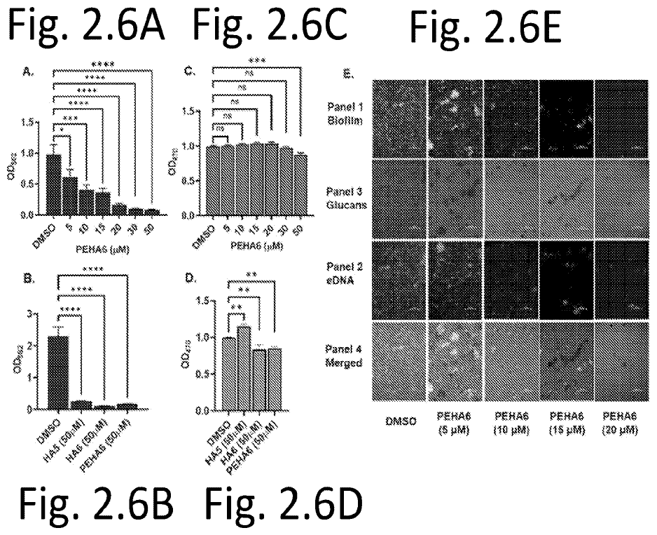

Aurones are a class of organic compounds that are gaining interest in medicinal chemistry due to their biological activities and presence in natural products³⁸. Aurone natural products play an important role in the pigmentation of flowers and fruits³⁹. Their reported bioactivities range from antifungal activity⁴⁰, antifeedant activity⁴¹, tyrosinase inhibition⁴², and antioxidant activity⁴³. In vitro antimicrobial activities of aurones and chalcones are widely reported⁴⁴. Biosynthetically, aurones are derived from chalcones³⁹. Therefore, a biomimetic synthetic approach was taken (Scheme 1, FIG. 1.2B) to generate a small library of aurones (FIG. 2), which includes one aurone derivative with an unsubstituted phenyl ring (MA1), five methoxy substituted aurones (MA2-6) and five hydroxy substituted aurones (HA2-6). These aurones were prepared from 2-hydroxychalcones (3a-f), which in turn were prepared by the Claisen-Schmidt aldol condensation⁴⁵ of the benzaldehydes (2a -f) and 2-hydroxyacetophenone (1) in the presence of KOH in ethanol in 37-90% yield. Cyclization of chalcones (3a-f) in the presence of Hg(OAc)₂ in anhydrous pyridine afforded the aurones (MA1-6) in 79-100% yield. Methyl groups in methoxyaurones (MA2-6) were then removed by treatment with BBr₃ in anhydrous CH₂Cl₂ to afford the hydroxyaurones (HA2-6) in 80-86% yield.

Solubility of MA1-6 and HA2-6 were determined as reported⁴⁶ (Table 1). As expected, the majority of aurone derivatives had better solubility than the lead compound IIIC5 (25 μg/mL)¹⁹. Among the aurones, hydroxy aurones were found to be more soluble than methoxy aurones and a trend of increasing solubility was observed with the increase in the number of hydroxy groups on the phenyl ring. The hydroxyaurone, HA5 with 2,4,5-trihydroxyphenyl ring was found to be the most soluble analog with the solubility of 120.09 μg/mL. A close analog, HA6 with 3,4,5-trihydroxyphenyl ring had the next highest solubility (90.77 μg/mL). The monohydroxyphenyl analog, HA2 showed the lowest solubility (18.93 μg/mL) among the hydroxyaurones. Methoxyaurones displayed a similar trend of increase in solubility with the increase in number of methoxy groups. The least soluble methoxyaurone was found to be the monomethoxy analog, MA2 with the solubility of 16.23 μg/mL. Trimethoxy aurone analogs, MA5 and MA6 were found to be the most soluble methoxyaurone analogs with the solubilities of 42.36 μg/mL and 44.68 μg/mL, respectively. The only exception to this trend was the 3,5-dimethoxy analog, MA4 which showed lower solubility of 18.97 μg/mL compared to 3,4-dimethoxy analog, MA3 (36.26 μg/mL). The aurone analogs that displayed lower solubility than IIIC5¹⁹ are MA1, MA2, MA4 and HA2.

TABLE 1
Reaction yields, solubility and biofilm inhibition profiles of 3a-f, MA1-6, and HA2-6.

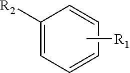

| Compd | R₂ | R₁ | Yield^{a} | Solubility^{b} | Biofilm IC₅₀^{c} |
|---|---|---|---|---|---|
| No | Group | Group | (%) | (ug/mL) | (UM) |
| IIIC5¹⁹ | | NA | NA | 25 ± 0.00 | 2.70 ± 0.09 |
| 3a 3b 3c 3d 3e 3f | 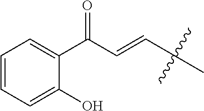 [SMILES: */C=C/C(=O)c1ccccc1O] | H 4-OMe 3,4-di-OMe 3,5-di-OMe 2,4,5-tri-OMe 3,4,5-tri-OMe | 62 37 53 78 90 85 | —^{d} —^{d} —^{d} —^{d} —^{d} —^{d} | >300^{e} >300^{e} >300^{e} >300^{e} >300^{e} 180.80 ± 0.65 |
| MA1 MA2 MA3 MA4 MA5 MA6 | 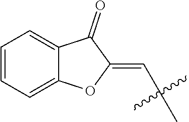 [SMILES: */C=C1\Oc2ccccc2C1=O] | H 4-OMe 3,4-di-OMe 3,5-di-OMe 2,4,5-tri-OMe 3,4,5-tri-OMe | 78 93 100 89 99 89 | 8.51 ± 2.00 16.23 ± 0.41 36.26 ± 1.56 18.97 ± 2.33 42.36 ± 2.86 44.68 ± 0.87 | 33.61 ± 0.53 107.80 ± 0.65 49.40 ± 4.79 >300^{e} 52.81 ± 7.42 >300^{e} |
| HA2 HA3 HA4 HA5 HA6 | 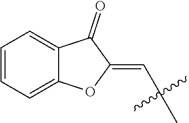 [SMILES: */C=C1\Oc2ccccc2C1=O] | 4-OH 3,4-di-OH 3,5-di-OH 2,4,5-tri-OH 3,4,5-tri-OH | 89 92 80 83 86 | 18.93 ± 1.41 81.56 ± 2.90 76.50 ± 0.57 120.09 ± 1.73 90.77 ± 0.48 | 18.79 ± 2.36 30.67 ± 2.28 94.22 ± 2.18 6.42 ± 0.61 18.92 ± 0.39 |
| G43²¹ | NA | NA | NA | NA | 6.28 ± 0.58 |
| Salicylic | NA | NA | NA | 1880 ± 30 | NA |
| acid⁴⁷ | | | | | |

^{a}Isolated yield and the compounds are fully characterized with ¹H-NMR, ¹³C-NMR and HRMS;
^{b}Solubility in water containing 1% DMSO determined by UV spectroscopy;
^{c}S. mutans UA159 were co-incubated with the compounds at various concentrations and biofilm formation was measured at OD562 using an established crystal violet protocol ⁴⁸. IC₅₀ values represent the means ± standard error mean (SEM) from three independent experiments;
^{d}Not determined;
^{e}Highest concentration tested.

Inhibition of S. mutans UA159 planktonic growth. In order to identify inhibitors of cariogenic biofilm without affecting the growth of oral bacteria, an evaluation was done on the effects of compounds 3a-e, MA1-6 and HA2-6 on S. mutans planktonic growth at a single concentration of 50 μM⁴⁸. No significant inhibition of planktonic growth was observed between the control group and treated groups for all chalcone derivatives, 3a-f (FIG. 1.3A). Methoxyaurones, MA2-6 were found to be slightly more bactericidal than chalcones showing 25-40% planktonic growth inhibition (FIG. 1.3B). The aurone analog with unsubstituted phenyl ring (MA1) showed the highest bactericidal activity with 80% inhibition of the planktonic growth. Some of the hydroxyaurones were more bactericidal than chalcones and methoxyaurones with HA2, HA3 and HA4 showing 60%, 40% and 30% inhibition, respectively. Two hydroxyaurones, HA5 and HA6 did not inhibit the planktonic growth of S. mutans at 50 μM and appeared to be promising lead compounds (FIG. 1.3C) for further evaluation.

Inhibition of S. mutans UA159 biofilms. Initial screening of compounds 3a-f, MA1-6 and HA2-6 in a single species S. mutans biofilm assay was carried out at a single treatment dose of 50 μM. Members of all three series of compounds were effective in inhibiting biofilms with hydroxyaurones exhibiting most pronounced activity compared to methoxyaurones and chalcones (FIG. 4). More importantly, all compounds showed varying degrees of selectivity towards inhibition of biofilm as opposed to growth. Chalcones, 3a-f were generally less active compared to aurones (FIG. 1.4A). The most active chalcone derivative, 3f exhibited 40% biofilm inhibition and no growth inhibition at 50 μM. The most potent methoxyaurone, MA5, exhibited 60% biofilm inhibition (FIG. 1.4B). However, this compound also inhibited 30% of bacterial growth at 50 μM making it a less selective biofilm inhibitor. The other methoxyaurones, MA1, MA2, MA4 and MA6 were relatively less active displaying only 20-40% biofilm inhibition, while MA3 was inactive at this dose. Overall, hydroxy aurones were better biofilm inhibitors than chalcones and methoxyaurones with derivatives, HA2, HA5 and HA6 showing more than 95% inhibition and HA3 showing about 80% inhibition of biofilms (FIG. 1.4C). Among the most active hydroxyaurones, 4-hydroxy analog, HA2 inhibited bacterial growth by 70% at the treated dose, making it a less selective biofilm inhibitor (FIG. 1.3C). The 3,5-dihydroxy aurone analog, HA4 did not show significant biofilm inhibition. The 2,4,5-trihydroxy and 3,4,5-trihydroxy analogs, HA5 and HA6, respectively were found to be the most active hydroxyaurone analogs with more than 95% biofilm inhibition and no effect on growth at 50 μM, making them the most active and selective biofilm inhibitors from this screening (FIG. 1.4C).

Inhibition of commensal Streptococci biofilms by hydroxyaurones. To determine the selectivity of the hydroxyaurones (HA2-6) toward S. mutans biofilm formation over the biofilms of commensal species, an evaluation was done on the effects of these compounds on the biofilm formation by two oral commensal Streptococci bacteria: S. gordonii and S. sanguinis. At 50 mM concentration, S. gordonii biofilm formation was inhibited by 40-60% (FIG. 1.5A), while S. sanguinis biofilm was inhibited by 30-40% (FIG. 1.5B). However, these effects were less pronounced than their effects on S. mutans biofilm. For example, compounds HA5 and HA6 displayed about 95% inhibition of S. mutans biofilm at 50 μM (FIG. 1.4C). These effects were also comparable to the control Gtf inhibitor G43 reported from our lab previously. In addition, a side-by-side comparison of biofilm inhibitory effects of 25 μM HA5 inhibited 80% of S. mutans biofilm while it did not significantly reduce S. Sanguinis biofilm and inhibited about 20% of S. gordonii biofilm (FIG. 1.8G). Overall, this data suggests that hydroxyaurones have a high degree of selectivity towards inhibiting pathogenic biofilms compared to commensal biofilms.

Considering the potential of methoxyaurones and hydroxyaurones for further development, their biofilm inhibitory activities were further characterized in serial dilutions and IC₅₀ values were determined. The hydroxyaurones were found to have lower IC₅₀ values compared to the corresponding methoxyaurones (Table 1). Among methoxyaurones, 3,4-dimethoxyaurone, MA3 was found to be the most active analog with an IC₅₀ value of 49.40 μM. The 2,4,5-trimethoyaurone, MA5 had a similar IC₅₀ value of 52.81 μM and the 4-methoxyaurone, MA2 had an IC₅₀ value of 107.80 while 3,4-dimethoxy and 3,4,5-trimethoxy aurones were inactive. Interestingly, the unsubstituted aurone, MA1 was more potent than all methoxy aurones with an IC₅₀ value of 33.61 μM. However, MA1 also displayed about 80% inhibition of S. mutans growth at 50 μM, suggesting that its observed biofilm inhibition may be arising from its bactericidal activity.

Two of the hydroxyaurones, 2,4,5-trihydroxy aurone (HA5) and 3,4,5-trihydroxy aurone (HA6) were found to be the most active derivatives with IC₅₀ values of 6.42 μM and 18.92 μM respectively. The 3,4-dihydroxyaurone, HA3 and 3,5-dihydroxyaurone, HA4 were found to be less active with IC₅₀ values of 30.67 μM and 94.22 μM, respectively. Among these, HA4 with no OH at the 4-position, was less active than HA3 with an OH group at 4-position. Interestingly, the monohydroxy analog, HA2 with an OH group at 4-postion was found to be more active than the dihydroxyaurones, HA3 and HA4. It should be noted that both of our most active analogs HA5 and HA6 also contained an OH group at the 4-position, indicating the importance of the 4-OH group for the biofilm inhibitory activities of hydroxyaurones. This observation is further supported by our co-crystal structure of HA5 in the GtfB active site, showing that the two oxygen atoms at the 4,5-position of the 2,4,5-trihydroxyphenyl moiety interacted with the key amino acid residues in the active site through the coordination with a conserved Ca²⁺ ion (FIG. 1.7). Of all the aurone analogs synthesized, 2,4,5-trihydroxyaurone, HA5 (FIG. 1.6E) was selected as our lead compound for further analysis and encapsulation studies based on its potent biofilm inhibition, lack of growth inhibition and improved solubility.

HA5 inhibits S. mutans UA159 biofilms, glucan production and eDNA levels. The antibiofilm activities of HA5 were further investigated by fluorescence microscopy imaging. Compound HA5 displayed a dose-dependent inhibition of S. mutans biofilm as shown in FIG. 1.6A. Staining of bacterial cells within biofilms with Syto-9 showed significant reduction in biofilms at 5 μM of HA5 and a complete inhibition at 50 μM of HA5 (FIG. 1.6C, Panel-I). The presence of glucans, which were stained with Cascade Blue-dextran conjugated dye, was significantly reduced at 5 μM of HA5 and no glucan formation was evident at 50 μM of HA5 (FIG. 1.6C, Panel II). In addition, propidium iodide was used to determine the presence of extracellular DNA (eDNA) in S. mutans biofilms. Again, there was a noticeable reduction of eDNA at 5 μM of HA5 and almost complete absence of eDNA at 50 μM of HA5 (FIG. 1.6C, Panel III). These findings reaffirm that HA5 inhibited S. mutans biofilms by preventing the synthesis of glucans and minimizing the presence of eDNA, two integral biofilm matrix elements crucial for S. mutans biofilm formation.

HA5 Inhibits the Glucan Production of S. mutans UA159 in a Dose Dependent Manner.

The interspecies co-adherence between S. mutans and other microorganisms in the oral cavity is critical for biofilm formation and cariogenicity. Though the mechanisms of such adhesions and co-aggregations are not fully elucidated, it is believed that the extracellular polysaccharide (EPS) matrix of S. mutans has an important role in this process^{49, 50}. It is reported that glucans synthesized by Gtfs when incorporated into the tooth pellicle to provide enhanced binding sites for other microorganisms to form stable and persistent microcolonies, which provides mechanical stability to the EPS matrix^{50, 51}. Therefore, Gtf inhibition assays were performed to assess the ability of HA5 to inhibit the Gtfs and glucan production using a reported procedure and IC₅₀ value was calculated⁵². Compound HA5 exhibited dose dependent inhibition of glucan production by Gtfs with an IC₅₀ value of 10.56 μM (FIG. 1.6B). These findings reinforce the biofilm inhibitory activity of HA5 and suggest that the compound inhibits biofilm formation by inhibiting glucans production by S. mutans Gtfs.

HA5 does not affect the growth of commensal streptococcal species. To determine if compound HA5 only selectively inhibits S. mutans biofilms over the growth of S. mutans and oral commensal species, the effects of HA5 on the growth of two representative commensal oral streptococci, S. gordonii and S. sanguinis, along with S. mutans at 50 μM and 100 μM doses were evaluated. As shown in FIG. 1.6D, compound HA5 did not inhibit the growth of two commensals compared to the control group at these doses that are much higher than its biofilm IC₅₀ value of 6.42 μM. Similarly, the compound did not inhibit S. mutans growth at these doses, suggesting that HA5 selectively inhibited S. mutans biofilms without affecting its growth as well as the growth of commensal species, S. gordonii and S. sanguinis (FIG. 1.6D).

HA5 is a polyhydroxy compound that contains a Michael acceptor functionality which raises concerns about non-specific and covalent binding. However, it is unlikely that HA5 is influenced by these mechanisms because our HA5/GtfB co-crystal structure clearly shows its binding in the catalytic site of GtfB with specific interactions with the Ca²⁺ ion and with active site residues and it does not show any covalent bond to its Michael acceptor site. To further validate that HA5 is not a Michael acceptor, the Gtf inhibition IC₅₀ values for HA5 in the presence and absence of a nucleophilic reagent, beta-mercaptoethanol (BME, 1 mM) have been determined and shown that BME doesn't reduce the Gtf inhibitory activity (7.84 μM vs 10.56 M)⁶¹. In addition, the Gtf inhibition IC₅₀ values for HA5 in the presence and absence of a detergent Triton-X-100 have been determined to show that it is not a non-specific inhibitor⁶². Triton-X-100 did not reduce the Gtf inhibitory activity (5.16 μM vs 10.56 μM) of HA5 suggesting that the observed Gtf inhibition is not due to non-specific binding.

Hydrogel encapsulated biofilm inhibitors. Hydrophilicity, the ease of chemical modification and structural stability of hydrogel matrices ensure excellent biocompatibility and versatility for its use in biomedical applications. Poly(methacrylic acid) [PMAA] hydrogel is an excellent platform for the pH-triggered drug delivery of the biofilm inhibitors as these respond to varying pH due to the existence of ionizable pendant groups (e.g. —COOH and —NH₂) in the network. In our previous studies, PMAA hydrogels have been prepared by layer-by-layer (LbL) assembly of hydrogen-bonded polymers of PMAA and poly(N-vinylpyrrolidone) (PVPON). The PMAA and PVPON layers were alternatingly adsorbed onto surfaces of porous inorganic microparticles of manganese oxide, followed by chemical crosslinking of PMAA with ethylenediamine and dissolution of the manganese oxide template microparticles⁶³⁻⁶⁵. The nanoscale multilayers of chemically crosslinked PMAA result in the interconnected porous hydrogel structure, which provides excellent drug loading capacity. Besides, the pH-responsiveness of the hydrogel can be easily tuned during particle formation by using pH-sensitive cross-linkers⁶⁶. The biocompatibility and degradability of hydrogel biomaterial has been demonstrated in the delivery of small-molecule drugs⁶⁵.

Encapsulation of HA5 inside (PMAA)₅ hydrogels microparticles. Compound HA5 was encapsulated in the (PMAA)₅ hydrogel cubes through post-loading by soaking the hydrogels in 5 mg/mL solution of HA5 in methanol for 48 h in the dark (FIGS. 1.7A-B). The free, non-encapsulated HA5 was removed from particle solution by rinsing with HEPES buffer (pH=7.4) five times using centrifugation at 5000 rpm for 10 min. The HA5 quantification was carried out with UV-visible spectroscopy (NanoDrop One C, ThermoFisher) at λ=448 nm using an HA5 calibration curve. The drug solution was analyzed before and after the exposure to the hydrogel particles and the differences in the absorbance spectra were used to determine the loading of the drug into the hydrogel network. The loading capacity was found to be 5.5×10⁻³ ng of HA5 per particle. To demonstrate the tooth adhesion of (PMAA) 5 hydrogel microparticles, a drop of the hydrogel particle dispersion was placed on the tooth surface and dried at room temperature for 10 min in a Petri dish and morphology of the hydrogels were analyzed using atomic force microscopy (AFM NTEGRA II microscope: NT-MDT) imaging. Freshly extracted, intact third molars with flat surfaces obtained from Dr. Nathaniel Lawson's lab (UAB School of Dentistry, IRB-300001291) were used in these studies. The AFM silicon probes NSG30 (NT-MDT, resonance frequency 240-440 kHz, force constant 22-100 N m⁻¹, tip radii is 10 nm, scan rate is 0.5 Hz) were used for imaging the tooth surfaces in tapping mode before and after hydrogel adhesion. The AFM image shows that the bare tooth surface displays natural topography (FIG. 1.7C) with height of 280 nm. After hydrogel addition, the cubical hydrogel particles are seen to adhere to the tooth surface (FIGS. 1.7D-E). The height of the dried hydrogel cubes was determined using section profiles, which indicated an average particle height of 1.3±0.2 μm. The hydrogel cubes decreased in size compared to their size in solution due to the hydrogel shrinkage upon drying 65.

Inhibition of biofilms and planktonic growth of by HEBI. Effects of HEBI and HA5 on biofilms and planktonic growth of S. mutans and two commensal Streptococci, S. gordonii and S. Sanguinis were evaluated at a single treatment dose of 25 μM. HEBI inhibited about 85-90% of S. mutans biofilms, which is comparable to 90% inhibition by HA5 (FIG. 1.7G). HEBI did not significantly inhibit the biofilms of commensal species S. gordonii and S. sanguinis at this concentration. As expected, based on our previous data, HEBI or HA5 did not affect the planktonic growth of S. mutans, or the commensal species S. gordonii and S. sanguinis at the treatment concentration of 25 μM (FIG. 1.7F).

Reduction of S. mutans virulence in vivo by HA5 or HEBI. The effects of compound HA5 and HEBI on S. mutans virulence were evaluated using a well-established gnotobiotic rat model of dental caries 67. Hydrogel microparticles with no drug were used to ensure that the observed anti-virulence activity observed with HEBI was not related to the hydrogel material. The standard NaF (250 ppm) was included as a positive control. A (vehicle+infection only) group was included as a negative control. All rats in the experimental groups and control groups were colonized with S. mutans UA159. A 4-week treatment of S. mutans UA159 infected gnotobiotic rats with 100 μM of HA5 or HEBI resulted in significant reduction in buccal and sulcal caries scores compared to control groups. Similar reductions in caries scores were also observed in proximal enamel caries scores (Table 3). An evaluation of the effect of the treatment on proximal dentinal scores was unable to be performed as there were no significant proximal dentinal lesions for the control and treated groups in this study. In comparison, the group treated with hydrogel (no drug) did not show any inhibition compared to the control group suggesting that the hydrogel as such has no antivirulence activity (Table 3). The observed reduction in caries scores by HA5 and HEBI were similar with HEBI displaying slightly better in vivo activity, possibly due to the pH-dependent slow release. The observed reduction in caries scores by HA5 or HEBI is lower than the 250 ppm NaF treatment. However, it should be noted that the concentration of NaF (250 ppm=5.95 mM) is about 59-fold higher than HA5 (100 μM). At the end of the study, the animals were euthanized, their mandibles excised for microbiological analysis of plaque samples on MS agar plates and BAP and for scoring of caries by the method of Keyes⁶⁸ to determine the bacterial colonization. The effect on bacterial colonization was not significant in HA5 or HEBI treated animals when compared to control group, while the bacterial colonization appears to be slightly reduced in unloaded hydrogel treated rats. This data suggests that HA5 and HEBI are less toxic to bacteria (Table 4). Moreover, the rats treated with the compound HA5 or HEBI did not experience any weight loss over the course of the study in comparison with the control group, suggesting that they are non-toxic (Table 4). Overall, our data suggest that HEBI can release HA5 in the rat's oral cavity under the acidic conditions of dental caries and the reduction in caries scores produced by HEBI is comparable to what is observed for HA5 treatment alone. These results also indicate that the compound HA5 or HEBI selectively target S. mutans virulence factors; Gtfs and Gtf-mediated biofilm formation, rather than a simple inhibition of bacterial growth and are very effective in inhibiting dental caries in vivo. All in vivo experimental protocols were approved by the University of Alabama at Birmingham Institutional Animal Care and Use Committee (protocol No: IACUC-20047). The methods were carried out in accordance with the relevant guidelines and regulations.

TABLE 3
Effect of HEBI or HA5 treatment on S. mutans UA159 induced dental caries.

| | | | | | | | | | Proximal |
|---|---|---|---|---|---|---|---|---|---|
| | | | | | | | | | Mean |
| | Buccal Mean | | | | Sulcal Mean | | | | Caries |
| | Caries Scores | | | | Caries Scores | | | | Scores |
| Treatment | (± SEM) | | | | (± SEM) | | | | (± SEM) |
| Group | E | Ds | Dm | Dx | E | Ds | Dm | Dx | E |
| UA159 | 14.6 ± 0.2 | 10.2 ± 0.4 | 6.0 ± 0.0 | 3.2 ± 0.5 | 25.6 ± 0.2 | 17.8 ± 0.2 | 10.4 ± 0.2 | 4.2 ± 0.2 | 6.0 ± 0.0 |
| untreated | | | | | | | | | |
| Hydrogel | 15.0 ± 0.3 | 9.6 ± 0.8 | 6.4 ± 0.7 | 3.8 ± 1.1 | 27.0 ± 0.5 | 19.6 ± 0.4 | 12.4 ± 0.9 | 6.0 ± 0.5 | 6.8 ± 0.5 |
| (no drug) | | | | | | | | | |
| HEBI | 7.4 ± 1.1 | 5.2 ± 1.2 | 2.4 ± 0.7 | 1.4 ± 0.6 | 16.0 ± 0.6 | 12.6 ± 0.2 | 6.8 ± 0.7 | 4.0 ± 0.0 | 3.6 ± 0.4 |
| (100 μM) | | | | | | | | | |
| HA5 | 11.0 ± 0.3 | 6.0 ± 0.4 | 3.8 ± 0.2 | 2.0 ± 0.7 | 22.0 ± 0.6 | 16.2 ± 0.4 | 8.8 ± 0.9 | 4.4 ± 0.9 | 4.0 ± 0.0 |
| (100 μM) | | | | | | | | | |
| NaF | 5.0 ± 0.7 | 2.4 ± 0.8 | 1.0 ± 0.8 | 0.0 ± 0.0 | 14.2 ± 0.7 | 11.2 ± 0.4 | 4.6 ± 0.2 | 1.0 ± 0.3 | 1.2 ± 0.8 |
| (250 ppm) | | | | | | | | | |

Enamel (E); Dentinal slight (Ds); Dentinal moderate (Dm); Dentinal extensive (Dx); Proximal dentinal scores are not included as there were no significant proximal dentinal lesions for the control and treated groups in this study.

TABLE 4
Effect of HEBI or HA5 treatment on S. mutans UA159 CFU
and the body weight of the animals.

| | | | Animals | |
|---|---|---|---|---|
| Treatment | CFU/mL (×10⁶) | | Weight | |
| Group | MS | BAP | (g) | Number |
| UA159 untreated | 4.2 ± 1.4 | 5.6 ± 1.6 | 141 ± 13 | 5 |
| Hydrogel (no drug) | 1.6 ± 0.5 | 2.4 ± 0.7 | 130 ± 9 | 5 |
| HEBI (100 μM) | 5.2 ± 1.4 | 5.5± 1.4 | 145 ± 12 | 5 |
| HA5 (100 μM) | 2.5 ± 0.8 | 3.0 ± 1.0 | 137 ± 13 | 5 |
| NaF (250 ppm) | 3.4 ± 0.5 | 4.0 ± 0.9 | 161 ± 13 | 5 |

Colony Forming Unit (CFU); Mitis Salivarius (MS); Blood Agar Plates (BAP).

### Conclusions

In conclusion, we have developed novel small-molecule inhibitors of S. mutans glucosyl transferases as selective biofilm inhibitors that do not affect the growth of oral commensal bacteria. The solubility and biofilm inhibitory activities of the lead compound were optimized for drug-encapsulation. The optimized lead compound, HA5 inhibited S. mutans biofilm with an IC₅₀ value of 6.42 μM without affecting its growth. Compound HA5 was further evaluated for its effect on the growth of oral commensal bacterial species S. gordonii and S. sanguinis and showed that it does not inhibit the growth of S. gordonii and S. sanguinis at 100 μM, which is 14-fold higher dose than its biofilm IC₅₀ value. The binding of HA5 to the glucosyl transferase, GtfB has been demonstrated by resolving a high-resolution X-ray co-crystal structure of HA5 with the catalytic domain of GtfB and mapped out its active site interactions. Compound HA5 inhibited S. mutans Gtfs and glucan production with an IC₅₀ value of 10.56 M in a Gtf inhibition assay. Compound HA5 was encapsulated into pH-responsive hydrogel microparticles to generate a hydrogel encapsulated biofilm inhibitor (HEBI), which displayed selective inhibition of S. mutans biofilm similar to HA5. The effects of HA5 or HEBI on the biofilm on commensal species, S. gordonii and S. sanguinis were minimal at 25 μM. A 4-week treatment of S. mutans UA159 infected gnotobiotic rats with 100 μM of HA5 or HEBI resulted in significant reduction in buccal, sulcal, and proximal dental caries scores compared to control groups demonstrating their antivirulence activities in vivo without affecting the bacterial colonization significantly. The rats treated with the HA5 or HEBI did not experience any weight loss over the course of the study in comparison with the control group, suggesting that the compound and material are non-toxic. Overall, our in vivo data suggests that HEBI can release HA5 in the rat oral cavity under the acidic conditions of dental caries infection and the reduce dental caries and the results are comparable to what is observed for HA5 treatment alone. Overall, the results of this study suggest that compound HA5 or HEBI selectively targeted S. mutans Gtfs and Gtf-mediated biofilm formation, rather than a simple inhibition of bacterial growth, demonstrating the potential of this compound and material to be developed further as novel dental caries treatments.

### EXPERIMENTAL

General considerations. ¹H-NMR and ¹³C-NMR spectra were recorded on Bruker Avance Neo 400 and Avance II 700 spectrometers using TMS or appropriate solvent signals as internal standard. The chemical shift values are given in parts per million (ppm) relative to the internal standard used and the coupling constants (J) are given in hertz (Hz). High resolution mass spectra (HRMS) were recorded using Waters AutoSpec-Ultima™ NT magnetic sector mass spectrometer with Electron Impact (EI) Ionization source. The mass analyzer is an electric-magnetic-electric (EBE) sector (a double focusing sector). Anhydrous solvents used for reactions were purchased in Sure-Seal™ bottles from Aldrich chemical company. Other chemical reagents were purchased from Aldrich or Fisher chemical companies and used as received. Reactions were monitored with thin layer chromatography (TLC), which was done on silica gel plates with fluorescent indicator (Silicycle, silica gel, UV254, 25 μm plates). The TLC spots were observed under UV light with the wavelengths 254 nm and 365 nm. The reaction mixtures were purified by column chromatography using Si gel (32-63 μm) from Dynamic Absorbent, Inc. Melting points were determined on a Mel-Temp II melting point apparatus and are uncorrected. All tested compounds have ≥95% purity as determined by HPLC. HPLC traces were obtained using Shimadzu SPD-M20A. Solubility of compounds were determined by UV-spectroscopy method using Agilent Cary 60 UV-Vis spectrophotometer. HPLC analysis of the final compounds were conducted using Kinetex 5 μm C18 100 Å, LC Column 150×4.6 mm, compound=3 mM, 20 μL injection, solvent: mobile phase buffer, Conditions: 60% MeCN/40% H₂O/0.1% Formic acid (isocratic), HPLC method 0-10 min, Signals were analyzed using a 254 nm UV detector. A chromatogram of Mobile Phase Buffer (20 μL) was obtained for comparison.

Poly(ethyleneimine) (PEI, average M_{w} 25000), ethylenediamine (EDA), manganese sulfate monohydrate, ammonium bicarbonate and 1-Ethyl-3-(3-(dimethylamino) propyl)-carbodiimide hydrochloride were purchased from Sigma-Aldrich. Poly(methacrylic acid) (PMAA, average M_{w} 22000 g mol⁻¹, Ð=1.3) were purchased from Fisher Scientific. Ultrapure de-ionized (DI) water with a resistivity of 18.2 MΩ-cm at 25° C. was used in all experiments. Monobasic and dibasic sodium phosphate (Fisher Scientific) were used for preparation of polymer and buffer solutions. Poly(N-vinylpyrrolidone) (PVPON, M_{w} 10000 g mol⁻¹) was from Sigma-Aldrich. Slices of human teeth were provided by Dr. Nathaniel Lawson (UAB School of Dentistry, IRB-300001291) and used as received.

The bacterial strains, S. mutans UA159, S. gordonii DL1, and S. sanguinis SK36 were inoculated statically at 37° C. under 5% CO₂ in Todd Hewitt Broth (THB) for 24 h. The cultures were then diluted with fresh THB (1:5) and reinoculated until optical density at 470 nm (OD₄₇₀) reached 1. The optical density was read using BioTek 800TS microplate reader at 470 nm for bacterial growth and 562 nm for biofilm stained with crystal violet. Data was plotted in Graphpad Prism9.

General procedure for the synthesis of chalcones (3a-f). To a solution of the 2-hydroxyacetophenone 1 (1 mmol) and benzaldehyde 2a (1 mmol) in EtOH (10 mL), an aqueous solution of KOH (40%, 1 mL) was added, and the reaction mixture was stirred at room temperature for 12 h. TLC examination (30% EtOAc in hexanes) indicated the completion of the reaction. The reaction mixture was then poured over crushed ice and acidified the pH to 2 using 1.0 N HCl. The precipitate formed was filtered, washed with copious amounts of water, and dried to obtain the crude product, which was purified on column chromatography over Si gel using 10% EtOAc in hexanes as eluent to afford clean chalcones 3a-f. All chalcone products were characterized by ¹H NMR, ¹³C NMR and HRMS as follows.

1-(2-Hydroxyphenyl)-3-phenyl-2-propen-1-one (3a): 61.9% yield, yellow solid; mp. 89-90° C.; ¹H NMR (700 MHz, CDCl₃) δ: 12.84 (s, 1H), 7.94-7.92 (m, 2H), 7.68-7.66 (m, 3H), 7.52-7.49 (m, 1H), 7.45 (t, 3H, J=3.1 Hz), 7.04 (d, 1H, J=8.5 Hz), 6.95 (t, 1H, J=7.6 Hz); ¹³C NMR (700 MHz, CDCl₃) δ: 193.7, 163.6, 145.5, 136.4, 134.6, 130.9, 129.7, 129.0, 128.7, 120.1, 120.0, 118.9, 118.6; HRMS [M−H]− calculated for C₁₅H₁₂O₂ 223.0759, found 223.0763.

1-(2-Hydroxyphenyl)-3-(4′-methoxyphenyl)-2-propen-1-one (3b): 36.5% yield, yellow solid; mp. 93-95° C.; ¹H NMR (700 MHz, CDCl₃) δ: 12.96 (s, 1H), 7.93-7.89 (m, 2H), 7.63 (d, 2H, J=8.6 Hz), 7.54 (d, 1H, J=15.4 Hz), 7.49 (t, 1H, J=7.7 Hz), 7.02 (d, 1H, J=8.3 Hz), 6.96-6.93 (m, 3H), 3.86 (s, 3H); ¹³C NMR (700 MHz, CDCl₃) δ: 193.7, 163.6, 162.0, 145.4, 136.2, 130.6, 129.5, 127.3, 120.1, 118.8, 118.6, 117.6, 114.5, 55.5; HRMS [M+H]⁺ calculated for C₁₆H₁₄O₃ 255.1021, found 255.1014.

3-(3′,4′-Dimethoxyphenyl)-1-(2-hydroxyphenyl) prop-2-en-1-one (3c): 53.0% yield, yellow solid; mp. 115-117° C.; ¹H NMR (400 MHz, CDCl₃) δ: 12.94 (s, 1H), 7.94 (dd, 1H, J=8.1, 1.6 Hz), 7.89 (d, 1H, J=15.4 Hz), 7.53 (d, 1H, J=15.4 Hz), 7.52-7.47 (m, 1H), 7.29-7.26 (m, 1H), 7.18 (d, 1H), 7.03 (dd, 1H, J=8.3 Hz), 6.97-6.91 (m, 2H), 3.97 (s, 3H), 3.95 (s, 3H); ¹³C NMR (700 MHz, CDCl₃) δ: 193.6, 163.6, 151.9, 149.4, 145.8, 136.3, 129.6, 127.7, 123.7, 120.2, 118.8, 118.7, 117.8, 111.2, 110.3, 56.1 (2); HRMS [M−H]⁺ calculated for C₁₇H₁₆O₄ 283.0970, found 283.0969.

3-(3′,5′-Dimethoxyphenyl)-1-(2-hydroxyphenyl) prop-2-en-1-one (3d): 78.0% yield, yellow solid; mp. 107-109° C. ¹H NMR (700 MHz, CDCl₃) δ: 12.80 (s, 1H), 7.90 (d, 1H, J=7.7 Hz), 7.81 (d, 1H, J=15.4 Hz), 7.58 (d, 1H, J=15.4 Hz), 7.49 (t, 1H, J=8.4 Hz), 7.02 (d, 1H, J=8.4 Hz), 6.93 (t, 1H, J=7.7 Hz), 6.77 (d, 2H, J=1.4 Hz), 6.56 (t, 1H, J=1.4 Hz), 3.83 (s, 6H); ¹³C NMR (700 MHz, CDCl₃) δ: 193.6, 163.5, 161.0, 145.4, 136.4, 136.4, 129.6, 120.5, 119.9, 118.8, 118.6, 106.5, 103.0, 55.4; HRMS [M−H]⁺ calculated for C₁₇H₁₆O₄ 283.0970, found 283.0969.

1-(2-Hydroxyphenyl)-3-(2′,4′,5′-trimethoxyphenyl) prop-2-en-1-one (3e): 90.0% yield, orange solid; mp. 135-137° C.; ¹H NMR (700 MHz, CDCl₃) δ: 13.08 (s, 1H), 8.21 (d, 1H, J=15.4 Hz), 7.91 (dd, 1H, J=7.7, 7.0 Hz), 7.60 (d, 1H, J=15.4 Hz), 7.46 (dd, 1H, J=8.4, 7.0 Hz), 6.99 (d, 1H, J=8.3 Hz), 6.92 (t, 1H, J=7.9 Hz), 6.5 (s, 1H), 3.94 (s, 3H), 3.92 (s, 3H), 3.90 (s, 3H); ¹³C NMR (700 MHz, CDCl₃) δ: 194.0, 163.5, 155.1, 152.9, 143.2, 140.9, 135.8, 129.5, 120.2, 118.6, 118.4, 117.7, 115.1, 111.7, 96.6, 56.5, 56.2, 56.0; HRMS calculated for C₁₈H₁₈O₅ 314.1154, found 314.1151.

1-(2-Hydroxyphenyl)-3-(3′,4′,5′-trimethoxyphenyl) prop-2-en-1-one (3f): 85.4% yield, yellow solid; mp. 155-157° C.; ¹H NMR (700 MHz, CDCl₃) δ: 12.86 (s, 1H), 7.92 (d, 1H, J=7.9 Hz), 7.83 (d, 1H, J=15.3 Hz), 7.53 (d, 1H, J=15.3 Hz), 7.49 (t, 1H, J=7.7 Hz), 7.02 (d, 1H, J=8.2 Hz), 6.94 (t, 1H, J=7.5 Hz), 6.87 (s, 2H), 3.93 (s, 6H), 3.91 (s, 3H); ¹³C NMR (700 MHz, CDCl₃) δ: 193.5, 163.6, 153.5, 145.6, 140.8, 136.4, 130.0, 129.6, 120.0, 119.2, 118.8, 118.6, 105.9, 61.0, 56.2; HRMS [M−H]⁺ calculated for C₁₈H₁₈O₅ 313.1076, found 313.1082.

General procedure for the synthesis of methoxy aurones (MA1-6). Chalcones 3a-f (0.6 mmol) was added to a homogeneous solution of Hg(OAc)₂ (0.221 g, 0.7 mmol) in anhydrous pyridine (20 mL) and the reaction mixture was heated at 110° C. for 12 h. The completion of reaction was marked by consumption of starting material and formation of a single product as visualized by TLC (50% EtOAc in hexanes). The reaction mixture was then quenched with ice and acidified to the pH of 2 by adding 1.0 N HCl. It was extracted in EtOAc (4×50 mL), and the combined extract was washed with water (2×50 mL), brine (1×50 mL), and dried over anhydrous Na₂SO₄. The drying agent was filtered off and the filtrate was concentrated in vacuo to obtain pure solid products MA1-6. All products were characterized by ¹H NMR, ¹³C NMR and HRMS as follows.

2-(Phenylmethylidene)-2,3-dihydro-1-benzofuran-3-one (MA1): 78.8% yield, off-white or beige solid; mp. 110-111° C.; ¹H NMR (400 MHz, CDCl₃) δ: 7.92 (d, 2H, J=7.5 Hz), 7.81 (d, 1H, J=8.4 Hz), 7.66-7.64 (m, 1H), 7.46 (t, 2H, J=7.8 Hz), 7.42-7.40 (m, 1H), 7.34 (d, 1H, J=8.4 Hz), 7.22 (t, 1H, J=7.4 Hz), 6.90 (s, 1H); ¹³C NMR (700 MHz, CDCl₃) δ: 184.8, 166.2, 146.9, 136.9, 132.3, 131.6, 129.9, 128.9, 124.7, 123.5, 121.6, 113.1, 113.0; HRMS [M−H]⁺ calculated for C₁₅H₁₀O₂ 221.0603, found 221.0596.

2-[(4′-Methoxyphenyl)methylidene]-2,3-dihydro-1-benzofuran-3-one (MA2): 92.5% yield, yellow solid; mp. 140-142° C.; ¹H NMR (700 MHz, CDCl₃) δ: 7.88 (d, 2H, J=8.8 Hz), 7.79 (d, 1H, J=7.6 Hz), 7.64-7.62 (m, 1H), 7.31 (d, 1H, J=8.3 Hz), 7.20 (t, 1H, J=7.5 Hz), 6.97 (d, 2H, J=8.8 Hz), 6.88 (s, 1H), 3.86 (s, 3H); ¹³C NMR (700 MHz, CDCl₃) δ: 184.5, 165.8, 161.1, 145.9, 136.5, 133.4, 125.0, 124.5, 123.3, 121.9, 114.5, 113.4, 112.9, 55.4; HRMS [M−H]⁺ calculated for C₁₆H₁₂O₃ 251.0708, found 251.0701.

2-[(3′,4′-Dimethoxyphenyl)methylidene]-2,3-dihydro-1-benzofuran-3-one (MA3): quantitative yield, yellow solid; mp. 157-159° C.; ¹H NMR (700 MHz, CDCl₃) δ: 7.81 (d, 1H, J=7.5 Hz), 7.66-7.63 (m, 1H), 7.54 (d, 1H, J=1.7 Hz), 7.50 (dd, 1H, J=8.4, 1.7 Hz), 7.31 (d, 1H, J=8.3 Hz), 7.22 (t, 1H, J=7.4 Hz), 6.95 (d, 1H, J=8.3 Hz), 6.87 (s, 1H), 3.98 (s, 3H), 3.95 (s, 3H); ¹³C NMR (700 MHz, CDCl₃) δ: 184.5, 165.8, 150.9, 149.1, 146.0, 136.6, 126.1, 125.3, 124.6, 123.4, 122.0, 113.8, 113.7, 112.9, 111.3, 56.0 (2). HRMS [M−H]− calculated for C₁₇H₁₄O₄ 281.0814, found 281.0805.

2-[(3′,5′-Dimethoxyphenyl)methylidene]-2,3-dihydro-1-benzofuran-3-one (MA4): 89.0% yield, yellow solid; mp. 157-160° C.; ¹H NMR (400 MHz, CDCl₃) δ: 7.78 (d, 1H J=7.2 Hz), 7.66-7.62 (m, 1H), 7.30 (d, 1H, J=8.3 Hz), 7.20 (t, 1H, J=7.5 Hz), 7.07 (s, 1H), 7.06 (s, 1H), 6.79 (s, 1H), 6.51 (t, 1H, J=2.2 Hz), 3.84 (s, 6H); ¹³C NMR (400 MHz, CDCl₃): 184.8, 166.2, 160.9, 147.1, 137.1, 133.9, 124.8, 123.6, 121.7, 113.1, 113.0, 109.6, 102.4, 55.6; HRMS [M+H]⁺ calculated for C₁₇H₁₄O₄ 283.0970, found 283.0979.

2-[(2′,4′,5′-Trimethoxyphenyl)methylidene]-2,3-dihydro-1-benzofuran-3-one (MA5): 99.0% yield, yellow solid; mp. 240-242° C.; ¹H NMR (700 MHz, CDCl₃) δ: 7.91 (s, 1H), 7.80 (dd, 1H, J=7.6, 7.0 Hz), 7.63-7.61 (m, 1H), 7.46 (s, 1H), 7.28 (d, 1H, J=8.2 Hz), 7.20 (t, 1H, J=7.6 Hz), 6.51 (s, 1H), 3.96 (s, 3H), 3.95 (s, 3H), 3.90 (s, 3H); ¹³C NMR (700 MHz, CDCl₃) δ: 184.3, 165.4, 155.3, 152.4, 145.7, 143.2, 136.2, 124.5, 123.2, 122.2, 114.5, 113.1, 112.8, 108.0, 96.3, 56.6, 56.4, 56.0; HRMS calculated for C₁₈H₁₆O₅ 312.0998, found 312.0998.

2-[(3′,4′,5′-Trimethoxyphenyl)methylidene]-2,3-dihydro-1-benzofuran-3-one (MA6): 89.0%, yellow solid; mp. 182-183° C.; ¹H NMR (700 MHz, CDCl₃) δ: 7.82 (d, 1H, J=7.2 Hz), 7.68-7.63 (m, 1H), 7.31 (d, 1H, J=8.3 Hz), 7.24 (t, 1H, J=7.4 Hz), 7.19 (s, 2H), 6.84 (s, 1H), 3.95 (s, 6H), 3.93 (s, 3H); ¹³C NMR (700 MHz, CDCl₃) δ: 184.5, 165.9, 153.3, 146.4, 140.1, 136.8, 127.7, 124.7, 123.5, 121.7, 113.4, 112.9, 108.9, 61.0, 56.2; HRMS [M+H]⁺ calculated for C₁₈H₁₆O₅ 313.1076, found 313.1082.

General procedure for the synthesis of hydroxy aurones (HA2-6). The methoxy aurone MA2-6 (0.25 mmol, 1.0 eq) was dissolved in anhydrous CH₂Cl₂(15 mL) and cooled down to 0° C. BBr₃ (1 mmol, 4.0 eq) was added slowly to the reaction mixture under N₂ atmosphere and stirred. The reaction mixture was allowed to attain room temperature and stirring continued for 12 h. TLC examination (50% EtOAc in hexanes) revealed the completion of the reaction. The reaction mixture was then cooled to 0° C. and carefully quenched with slow drop-wise addition of water until the excess BBr₃ reacted completely. The precipitated solid product was filtered, washed with water, and dried over CaCl₂) in a vacuum desiccator. The crude product thus obtained was purified by column chromatography over Si gel using 10% MeOH in CH₂Cl₂ to afford pure hydroxyl aurones HA2-6. All hydroxy aurones were characterized by ¹H NMR, ¹³C NMR and HRMS as follows.

2-[(4′-Hydroxyphenyl)methylidene]-2,3-dihydro-1-benzofuran-3-one (HA2): 89.8% yield, yellow solid; mp. 264-266° C.; ¹H NMR (700 MHz, DMSO-d₆) δ: 10.23 (s, 1H), 7.88 (d, 2H, J=8.4 Hz), 7.79-7.77 (m, 2H), 7.54 (d, 1H, J=8.6 Hz), 7.30 (t, 1H, J=7.4 Hz), 6.91 (d, 3H, J=8.8 Hz); ¹³C NMR (700 MHz, DMSO-d6) δ: 183.2, 165.0, 159.8, 144.7, 137.2, 133.8, 124.1, 123.7, 122.9, 121.3, 116.2, 113.5, 113.2; HRMS [M+H]⁺ calculated for C₁₅H₁₀O₃ 239.0708, found 239.0718.

2-[(3′,4′-Dihydroxyphenyl)methylidene]-2,3-dihydro-1-benzofuran-3-one (HA3): 92.0% yield, yellow solid; mp. 231-233° C.; ¹H NMR (400 MHz, DMSO-d₆) δ: 9.82 (bs, 1H), 9.34 (bs, 1H), 7.80-7.76 (m, 2H), 7.53-7.50 (m, 2H), 7.34-7.28 (m, 2H), 6.86 (d, 1H), 6.82 (s, 1H); ¹³C NMR (700 MHz, DMSO-d₆) δ: 183.1, 165.0, 148.6, 145.7, 144.7, 137.1, 125.2, 124.1, 123.7, 123.3, 121.4, 118.3 (d), 116.1 (d), 114.0 (d), 113.0; HRMS [M+H]+ calculated for C₁₅H₁₀O₄ 255.0657, found 255.0660.

2-[(3′,5′-Dihydroxyphenyl)methylidene]-2,3-dihydro-1-benzofuran-3-one (HA4): 80.0% yield, grey solid; decomposed at 250° C.; ¹H NMR (700 MHz, DMSO-d₆) δ: 7.79 (t, 2H, J=7.4 Hz), 7.50 (d, 1H, J=8.6 Hz), 7.30 (t, 1H, J=7.3 Hz), 6.88 (d, 2H, J=2.3 Hz), 6.73 (s, 1H), 6.36 (t, 1H, J=2.0 Hz); ¹³C NMR (700 MHz, DMSO-d₆) δ: 183.7, 165.4, 158.7, 146.2, 137.8, 133.2, 124.4, 124.0, 121.0, 113.1, 113.1, 109.7, 105.0; HRMS [M−H]− calculated for C₁₅H₁₀O₄ 253.0501, found 253.0513.

2-[(2′,4′,5′-Trihydroxyphenyl)methylidene]-2,3-dihydro-1-benzofuran-3-one (HA5): 83.0% yield, red solid; mp. 191-193° C.; ¹H NMR (400 MHz, Acetone-d₆) δ: 8.94 (s, 1H), 8.71 (s, 1H), 7.97 (s, 1H), 7.85 (s, 1H), 7.71-7.76 (m, 2H), 7.43 (d, 1H, J=8.2 Hz), 7.40 (s, 1H), 7.28 (t, 1H, J=7.4 Hz), 6.57 (s, 1H); ¹³C NMR (400 MHz, Acetone-d₆) δ: 183.9, 166.1, 153.8, 150.7, 145.6, 139.7, 137.2, 124.7, 124.1, 123.0, 117.7, 113.7, 111.6, 108.9, 103.7; HRMS calculated for C₁₅H₁₀O₅ 270.0528, found 270.0529.

2-[(3′,4′,5′-Trihydroxyphenyl)methylidene]-2,3-dihydro-1-benzofuran-3-one (HA6): 86.0% yield, greenish-yellow solid; decomposed at 256° C.; ¹H NMR (700 MHz, DMSO-d₆) δ: 9.28 (s, 2H), 9.05 (s, 1H), 7.78-7.76 (m, 2H), 7.48 (d, 1H, J=8.5 Hz), 7.28 (dd, 1H, J=14.8, 7.3 Hz), 7.03 (s, 2H), 6.72 (s, 1H); ¹³C NMR (700 MHz, DMSO-d₆) δ: 183.0, 164.9, 146.2, 144.8, 137.1, 137.0, 124.2, 123.7, 122.0, 121.4, 114.5, 113.0, 111.3; HRMS [M−H]− calculated for C₁₅H₁₀O₅ 269.0450, found 269.0445.

Biofilm inhibition assays. Biofilm inhibition assays were performed in polystyrene microtiter 96 well plates. Stock solutions were prepared in chemically defined medium (CDM, JRH Biosciences, Lenexa, KS) with 2% sucrose, 1% bacteria cultures and various concentrations of the small molecule inhibitors to examine their activity against biofilm formation as described^{69, 70}. These stocks were assayed in 96 well plates in triplicate and incubated at 37° C. and 5% CO₂ for 16 h. After reading optical density for bacterial growth, the plate was gently washed with water, dried, and stained with crystal violet, and then gently rinsed again with deionized water leaving the stained biofilm at the bottoms of the wells. Biofilms was dissolved in 200 μL of 30% acetic acid and absorbance at 562 nm was used read to determine biofilm biomass. Each assay was carried out at least in triplicate. Biofilm inhibitory concentration (IC₅₀) of the compounds was determined by serial dilutions.

Gtf inhibition determined by glucan quantification assays. Overnight cultures of S. mutans UA159 were centrifuged (6500 rpm, 4° C., 10 min) to remove cells. Supernatant was mixed with ethanol (1:1) and incubated at −80° C. for 1 h. The precipitated Gtfs were palleted using centrifugation and resuspended in chemically defined media (CDM) 10 μL of Gtfs suspended in CDM were assayed on Ibidi slides with varying concentrations of inhibitor, 1% sucrose, 1% DMSO and 1 μM Cascade blue dye in CDM. The slides were then incubated at 37° C. with 5% CO₂ for 16 h after which, the wells of Ibidi slides were gently rinsed with 1×PBS and treated with 1×PBS for fluorescence microscopy imaging. The images obtained were processed in ImageJ to quantify glucans and graphed in GraphPad Prism.

S. mutans, S. gordonii, and S. sanguinis growth assays. Effects of compounds on S. mutans and commensal bacterial growth were evaluated using the growth assay as described⁶⁹. S. mutans UA159, S. gordonii DL1, S. sanguinis SK36, cultures were grown for 24 h under 5% CO₂ at 37° C. These cultures were then reinoculated with fresh THB (1:5) until OD₄₇₀=1 when the bacteria were ready to be used. Different concentrations of the inhibitor were assayed in chemically defined media (CDM) with 1% of the bacteria, 1% sucrose and 1% DMSO in 96 well plates. The 96 well plates were incubated under 5% CO₂ at 37° C. for 16 h. Growth of the bacteria was read after 16 h at OD₄₇₀. Each assay was carried out at least in triplicate.

Synthesis of porous cubic manganese oxide microparticle templates. Porous Mn₂O₃ microparticle templates of 3 μm in size were synthesized as described previously^{63, 65} Briefly, a nano-seed solution was prepared by mixing 0.04 g of NH₄HCO₃ and 0.02 g of MnSO₄ in DI water (200 mL). Then, the nano-seed solution (80 mL) was added to a 6 mM of MnSO₄ (1000 mL) followed by 6 mM solution of NH₄HCO₃(1000 mL) both containing 2-propanol (0.5% vol) was added to the nano-seed solution and was heated at 60° C. for 30 minutes to produce 3 μm cubic manganese carbonate particles. Once collected and dried via filtration, the Mn₂CO₃ microparticles were heated at 650° C. for 3.5 h in the muffled oven to produce porous Mn₂O₃ microparticles.

Synthesis of cubical hydrogel microparticles. pH-Responsive cubic hydrogel cubic microparticles were synthesized by depositing hydrogen-bonded [PMAA/PVPON]ₙ (the subscript denotes the number of polymer bilayers) multilayers at the surfaces of Mn₂O₃ microparticle templates. The porous templates were first exposed to an aqueous poly(ethyleneimine) (PEI) solution in deionized (DI) water (1.5 mg/mL) for 1 h to enhance the adsorption of the following (PMAA/PVPON) layers to the particle surfaces followed by deposition of the polymers from aqueous polymeric solutions (1.5 mg/mL) at pH=2 for 45 min each. The polymer deposition was achieved through sonication (15 min) and shaking (30 min) of the manganese oxide porous templates in polymer solutions. After the deposition of each layer, the template particles were centrifuged for 10 min at 4,900 rpm and re-suspended in phosphate buffer solution (0.01 M, pH=2) twice to rinse away excess polymer before the next deposition cycle. Following the deposition of a 5-bilayer (PMAA/PVPON)₅ coating, the PMAA layers were cross-linked with ethylenediamine by, first, activating the PMAA carboxylic groups with a carbodiimide solution (5 mg/mL, pH=5, 0.01 M phosphate) for 30 min, then exposing the particles to ethylenediamine (12 μL/mL in 0.01 M phosphate, pH=5.8) for 16 h. Afterwards, PVPON was removed from the PMAA network by exposing the core-shell particles to 0.01 M phosphate buffer solution (pH=8.5) for 24 h while shaking. Cubic PMAA hydrogel microparticles were obtained after dissolving the manganese oxide core in hydrochloric acid solution (8M HCl) for 24 h. The hydrogel microparticles were treated with ethylenediamine tetraacetic acid disodium salt solution (EDTA, 0.1 M) at pH=7 overnight by shaking to remove any residual manganese ions in the hydrogel network. The PMAA hydrogel microparticles were then purified by dialysis in DI water for 3 days using a Float-a-Lyzer (Fisher; MWCO 20 kDa).

Rat model of dental caries. In vivo studies of colonization and virulence of S. mutans were evaluated using a previously reported rat model of dental caries⁷¹. Offspring of gnotobiotic Fischer 344 rats used in this experiment were bred and maintained in trexler isolators. Male and female rat pups were removed from isolators at 20 days of age and randomly assigned into 5 treatment groups of 5 rats/group in cages with filter tops. Rats were then infected with S. mutans UA159 strain by oral swabbing daily for four consecutive days with a fresh overnight culture of S. mutans UA159. Rats were provided with caries promoting Teklad Diet 305 containing 5% sucrose (Harlan Laboratories, Inc., Indianapolis, IN) and sterile drinking water ad libitum. Oral swabs were taken 5 days post-infection and plated on Todd Hewitt (TH) agar plates and incubated at 37° C. in an environment of 5% CO₂ in the air to confirm colonization. Rats were weighed at weaning and at the termination of the experiment. One-week post-infection, the molars of the rats were treated topically twice daily for 4 weeks with the test compounds using camel-hair brushes. The five treatment groups used in this study were: 1) HEBI (100 μM); 2) HA5 (100 μM); 3) hydrogel encapsulated PBS (no drug) containing 0.1% DMSO (negative control), 4) 250 ppm NaF (positive control) and 5) infected untreated group (negative control). Drinking water was withheld for 60 min following each treatment with the compound. Animals were weighed at weaning and at the termination of the experiment. On day 60, the rats were sacrificed using CO₂ followed by cervical dislocation or bilateral thoracotomy. The mandibles were surgically removed and cleaned of excess tissue to assess the level of bacteria present and the extent of caries formation. The right mandibles from each rat were placed in a tube containing phosphate buffer (3 mL), placed on ice and sonicated (10 sec) to release bacteria from the molars. Each sample was serially diluted, plated on blood agar plates (BAP) and mitis-salivarius (MS) agar plates and incubated in an environment of 5% CO₂ at 37° C. to quantify the level of total bacteria and S. mutans present in the plaque. The right and left mandibles from each rat were then placed in 95% ethanol for 24 h. The mandibles will be cleaned and stained overnight with murexide solution. After drying, the mandibles were sectioned and scored for caries activity using the Keyes method⁶⁸. Caries scores were recorded for the buccal, sulcal and proximal molar surfaces individually so that differences among the surfaces can be distinguished. Statistical significance in the mean caries scores, colony-forming units (CFUs)/mandible and body weights between groups of rats were determined by one-way analysis of variance (ANOVA) with the Tukey-Kramer multiple comparison test using the InStat program (Graphpad Software, San Diego, CA). When determining the statistical significance between the two groups, an unpaired t-test was applied. Differences between groups were considered significant at a P-value<0.05. All experimental protocols were approved by the University of Alabama at Birmingham Institutional Animal Care and Use Committee (protocol No: IACUC-20047). The methods were carried out in accordance with the relevant guidelines and regulations.

### References for Example 1

(1) Marsh, P. D. Dental Plaque as a Microbial Biofilm. Caries Res. 2004, 38, 204-211.
(2) Vos, T., Allen, C., Arora, M., Barber, R. M., Bhutta, Z. A., Brown, A., Carter, A., Casey, D. C., Charlson, F. J., Chen, A. Z., Coggeshall, M., Cornaby, L., Dandona, L., Dicker, D. J., Dilegge, T., Erskine, H. E., Ferrari, A. J., Fitzmaurice, C., Fleming, T., . . . . Murray, C. J. L . . . Global, Regional, and National Incidence, Prevalence, and Years Lived with Disability for 310 Diseases and Injuries, 1990-2015: A Systematic Analysis for the Global Burden of Disease Study 2015. Lancet 2016, 388, 1545-1602.
(3) Banas, J. A.; Vickerman, M. M. Glucan-binding Proteins of the Oral Streptococci. Crit. Rev. Oral. Biol. Med. 2003, 14, 89-99. Kim, J. K.; Baker, L. A.; Davarian, S.; Crimmins, E. Oral Health Problems and Mortality. J. Dent. Sci. 2013, 8, 115-120.
(4) Hamada, S.; Slade, H. D. Biology, Immunology, and Cariogenicity of Streptococcus mutans. Microbiol. Rev. 1980, 44, 331-384. Loesche, W. J. Role of Streptococcus mutans in Human Dental Decay. Microbiol. Rev. 1986, 50, 353-380.
(5) Overman, P. R. Biofilm: A New View of Plaque. J. Contemp. Dent. Pract. 2000, 1, 18-29.
(6) Ahovuo-Saloranta, A.; Forss, H.; Walsh, T.; Nordblad, A.; Mäkela, M.; Worthington, H. V. Pit and Fissure Sealants for Preventing Dental Decay in Permanent Teeth. Cochrane Database Syst. Rev. 2017, 7, Cd001830. Baik, A.; Alamoudi, N.; El-Housseiny, A.; Altuwirqi, A. Fluoride Varnishes for Preventing Occlusal Dental Caries: A Review. Dent. J. 2021, 9, 1-15.
(7) Aoun, A.; Darwiche, F.; Al Hayek, S.; Doumit, J. The Fluoride Debate: The Pros and Cons of Fluoridation. Prev. Nutr. Food Sci. 2018, 23, 171-180. Duffin, S.; Duffin, M.; Grootveld, M. Revisiting Fluoride in the Twenty First Century: Safety and Efficacy Considerations. Front. Oral Health 2022, 3, 873157.
(8) Grandjean, P. Developmental Fluoride Neurotoxicity: An Updated Review. Environ. Health 2019, 18, 110.
(9) Oyanagi, T.; Tagami, J.; Matin, K. Potentials of Mouthwashes in Disinfecting Cariogenic Bacteria and Biofilms Leading to Inhibition of Caries. Open Dent. J. 2012, 6, 23-30.
(10) Kolenbrander, P. E. Oral Microbial Communities: Biofilms, Interactions, and Genetic Systems. Annu. Rev. Microbiol. 2000, 54, 413-437.
(11) Ren, Z.; Chen, L.; Li, J.; Li, Y. Inhibition of Streptococcus mutans Polysaccharide Synthesis by Molecules Targeting Glycosyltransferase Activity. J. Oral Microbiol. 2016, 8, 31095. Ren, Z.; Cui, T.; Zeng, J.; Chen, L.; Zhang, W.; Xu, X.; Cheng, L.; Li, M.; Li, J.; Zhou, X.; Li, Y. Molecule Targeting Glucosyltransferase Inhibits Streptococcus mutans Biofilm Formation and Virulence. Antimicrob. Agents Chemother. 2015, 60, 126-135. Taubman, M. A.; Nash, D. A. The Scientific and Public Health Imperative for a Vaccine Against Dental Caries. Nat. Rev. Immunol. 2006, 6, 555-563.
(12) Koo, H.; Jeon, J. G. Naturally Occurring Molecules as Alternative Therapeutic Agents Against Cariogenic Biofilms. Adv. Dent. Res. 2009, 21, 63-68.
(13) Jenkinson, H. F.; Lamont, R. J. Oral Microbial Communities in Sickness and in Health. Trends Microbiol. 2005, 13, 589-595. Kolenbrander, P. E.; Andersen, R. N.; Blehert, D. S.; Egland, P. G.; Foster, J. S.; Palmer, R. J., Jr. Communication Among Oral Bacteria. Microbiol. Mol. Biol. Rev. 2002, 66, 486-505. Kuramitsu, H. K.; He, X.; Lux, R.; Anderson, M. H.; Shi, W. Interspecies Interactions Within Oral Microbial Communities. Microbiol. Mol. Biol. Rev. 2007, 71, 653-670.
(14) Bowden, G. H.; Hamilton, I. R. Survival of Oral Bacteria. Crit. Rev. Oral Biol. Med. 1998, 9, 54-85. Quivey, R. G., Jr.; Kuhnert, W. L.; Hahn, K. Adaptation of Oral Streptococci to Low pH. Adv. Microb. Physiol. 2000, 42, 239-274.
(15) Matsumoto-Nakano, M. Role of Streptococcus mutans Surface Proteins for Biofilm Formation. Jpn. Dent. Sci. Rev. 2018, 54, 22-29.
(16) Yamashita, Y.; Bowen, W. H.; Burne, R. A.; Kuramitsu, H. K. Role of the Streptococcus mutans gtf Genes in Caries Induction in the Specific Pathogen-free Rat Model. Infect. Immun. 1993, 61, 3811-3817.
(17) Aoki, H.; Shiroza, T.; Hayakawa, M.; Sato, S.; Kuramitsu, H. K. Cloning of a Streptococcus mutans Glucosyltransferase Gene Coding for Insoluble Glucan Synthesis. Infect. Immun. 1986, 53, 587-594. Hanada, N.; Kuramitsu, H. K. Isolation and Characterization of the Streptococcus mutans gtfC Gene Coding for Synthesis of Both Soluble and Insoluble Glucans. Infect. Immun. 1988, 56, 1999-2005.
(18) Hanada, N.; Kuramitsu, H. K. Isolation and Characterization of the Streptococcus mutans gtfD Gene Coding for Primer-dependent Soluble Glucan Synthesis. Infect. Immun. 1989, 57, 2079-2085. Monchois, V.; Willemot, R. M.; Monsan, P. Glucansucrases: Mechanism of Action and Structure-function Relationships. FEMS Microbiol. Rev. 1999, 23, 131-151.
(19) Nijampatnam, B.; Ahirwar, P.; Pukkanasut, P.; Womack, H.; Casals, L.; Zhang, H.; Cai, X.; Michalek, S. M.; Wu, H.; Velu, S. E. Discovery of Potent Inhibitors of Streptococcus mutans Biofilm with Antivirulence Activity. ACS Med. Chem. Lett. 2021, 12, 48-55.
(20) Nijampatnam, B.; Casals, L.; Zheng, R.; Wu, H.; Velu, S. E. Hydroxychalcone Inhibitors of Streptococcus mutans Glucosyl transferases and Biofilms as Potential Anticaries Agents. Bioorg. Med. Chem. Lett. 2016, 26, 3508-3513. Nijampatnam, B.; Zhang, H.; Cai, X.; Michalek, S. M.; Wu, H.; Velu, S. E. Inhibition of Streptococcus mutans Biofilms by the Natural Stilbene Piceatannol Through the Inhibition of Glucosyltransferases. ACS Omega 2018, 3, 8378-8385.
(21) Zhang, Q.; Nijampatnam, B.; Hua, Z.; Nguyen, T.; Zou, J.; Cai, X.; Michalek, S. M.; Velu, S. E.; Wu, H. Structure-Based Discovery of Small Molecule Inhibitors of Cariogenic Virulence. Sci. Rep. 2017, 7, 5974.
(22) Butron Téllez Giron, C.; Hernandez Sierra, J. F.; DeAlba-Montero, I.; Urbano Pena, M. L. A.; Ruiz, F. Therapeutic Use of Silver Nanoparticles in the Prevention and Arrest of Dental Caries. Bioinorg. Chem. Appl. 2020, 2020, 8882930.
(23) Sims, K. R.; Maceren, J. P.; Liu, Y.; Rocha, G. R.; Koo, H.; Benoit, D. S. W. Dual Antibacterial Drug-loaded Nanoparticles Synergistically Improve Treatment of Streptococcus mutans Biofilms. Acta Biomater. 2020, 115, 418-431.
(24) Horev, B.; Klein, M. I.; Hwang, G.; Li, Y.; Kim, D.; Koo, H.; Benoit, D. S. pH-activated nanoparticles for controlled topical delivery of farnesol to disrupt oral biofilm virulence. ACS Nano 2015, 9, 2390-2404. Yi, Y.; Wang, L.; Chen, L.; Lin, Y.; Luo, Z.; Chen, Z.; Li, T.; Wu, J.; Zhong, Z. Farnesal-loaded pH-Sensitive Polymeric Micelles Provided Effective Prevention and Treatment on Dental Caries. J. Nanobiotech. 2020, 18, 89.
(25) Jailani, A.; Kalimuthu, S.; Rajasekar, V.; Ghosh, S.; Collart-Dutilleul, P. Y.; Fatima, N.; Koo, H.; Solomon, A. P.; Cuisinier, F.; Neelakantan, P. Trans-Cinnamaldehyde Eluting Porous Silicon Microparticles Mitigate Cariogenic Biofilms. Pharmaceutics 2022, 14, 1428.
(26) Liu, Y.; Huang, Y.; Kim, D.; Ren, Z.; Oh, M. J.; Cormode, D. P.; Hara, A. T.; Zero, D. T.; Koo, H. Ferumoxytol Nanoparticles Target Biofilms Causing Tooth Decay in the Human Mouth. Nano Lett. 2021, 21, 9442-9449. Liu, Y.; Naha, P. C.; Hwang, G.; Kim, D.; Huang, Y.; Simon-Soro, A.; Jung, H. I.; Ren, Z.; Li, Y.; Gubara, S.; Alawi, F.; Zero, D.; Hara, A. T.; Cormode, D. P.; Koo, H. Topical Ferumoxytol Nanoparticles Disrupt Biofilms and Prevent Tooth Decay In Vivo via Intrinsic Catalytic Activity. Nat. Commun. 2018, 9, 2920.
(27) Melo, M. A.; Guedes, S. F.; Xu, H. H.; Rodrigues, L. K. Nanotechnology-based Restorative Materials for Dental Caries Management. Trends Biotechnol. 2013, 31, 459-467.
(28) Mohire, N. C.; Yadav, A. V. Chitosan-based Polyherbal Toothpaste: As Novel Oral Hygiene Product. Indian J. Dent. Res. 2010, 21, 380-384.
(29) Seneviratne, C. J.; Leung, K. C.-F.; Wong, C.-H.; Lee, S.-F.; Li, X.; Leung, P. C.; Lau, C. B. S.; Wat, E.; Jin, L. Nanoparticle-Encapsulated Chlorhexidine against Oral Bacterial Biofilms. Plos One 2014, 9, e103234. Zhang, J. F.; Wu, R.; Fan, Y.; Liao, S.; Wang, Y.; Wen, Z. T.; Xu, X. Antibacterial Dental Composites with Chlorhexidine and Mesoporous Silica. J. Dent. Res. 2014, 93, 1283-1289.
(30) Nguyen, S.; Hiorth, M.; Rykke, M.; Smistad, G. Polymer Coated Liposomes for Dental Drug Delivery: Interactions with Parotid Saliva and Dental Enamel. Eur. J. Pharm. Sci. 2013, 50, 78-85.
(31) Feitosa, S. A.; Palasuk, J.; Kamocki, K.; Geraldeli, S.; Gregory, R. L.; Platt, J. A.; Windsor, L. J.; Bottino, M. C. Doxycycline-Encapsulated Nanotube-Modified Dentin Adhesives. J. Dent. Res. 2014, 93, 1270-1276.
(32) Zhou, Y.; Yang, J.; Lin, Z.; Li, J.; Liang, K.; Yuan, H.; Li, S.; Li, J. Triclosan-Loaded Poly(amido amine) Dendrimer for Simultaneous Treatment and Remineralization of Human Dentine. Colloids Surf. B. 2014, 115, 237-243.
(33) Naha, P. C.; Liu, Y.; Hwang, G.; Huang, Y.; Gubara, S.; Jonnakuti, V.; Simon-Soro, A.; Kim, D.; Gao, L.; Koo, H.; Cormode, D. P. Dextran-Coated Iron Oxide Nanoparticles as Biomimetic Catalysts for Localized and pH-Activated Biofilm Disruption. ACS Nano 2019, 13, 4960-4971.
(34) Aframian, D. J.; Davidowitz, T.; Benoliel, R. The Distribution of Oral Mucosal pH Values in Healthy Saliva Secretors. Oral Dis. 2006, 12, 420-423. Baliga, S.; Muglikar, S.; Kale, R. Salivary pH: A Diagnostic Biomarker. J. Indian Soc. Periodont. 2013, 17, 461-465.
(35) Lazarchik, D. A.; Filler, S. J. Effects of Gastroesophageal Reflux on the Oral Cavity. Am. J. Med. 1997, 103, 107S-113S.
(36) Markitziu, A.; Aframian, D. Gastro-Intestinal Disorders. Br. Dent. J. 1997, 182, 207. Robb, N. D.; Smith, B. G.; Geidrys-Leeper, E. The Distribution of Erosion in the Dentitions of Patients with Eating Disorders. Br. Dent. J. 1995, 178, 171-175.
(37) Meyer, F.; Enax, J.; Epple, M.; Amaechi, B. T.; Simader, B. Cariogenic Biofilms: Development, Properties, and Biomimetic Preventive Agents. Dent. J. 2021, 9, 88.
(38) Lawrence, N. J.; Rennison, D.; McGown, A. T.; Hadfield, J. A. The Total Synthesis of an Aurone Isolated from Uvaria hamiltonii: Aurones and Flavones as Anticancer Agents. Bioorg. Med. Chem. Lett. 2003, 13, 3759-3763.
(39) Ono, E.; Fukuchi-Mizutani, M.; Nakamura, N.; Fukui, Y.; Yonekura-Sakakibara, K.; Yamaguchi, M.; Nakayama, T.; Tanaka, T.; Kusumi, T.; Tanaka, Y. Yellow Flowers Generated by Expression of the Aurone Biosynthetic Pathway. Proc. Natl. Acad. Sci. 2006, 103, 11075-11080.
(40) Brooks, C. J. W.; Watson, D. G. Phytoalexins. Nat. Prod. Rep. 1985, 2, 427-459.
(41) Morimoto, M.; Fukumoto, H.; Nozoe, T.; Hagiwara, A.; Komai, K. Synthesis and Insect Antifeedant Activity of Aurones against Spodoptera litura Larvae. J. Agric. Food Chem. 2007, 55, 700-705.
(42) Okombi, S.; Rival, D.; Bonnet, S.; Mariotte, A. M.; Perrier, E.; Boumendjel, A. Discovery of Benzylidenebenzofuran-3(2H)-one (aurones) as Inhibitors of Tyrosinase Derived from Human Melanocytes. J. Med. Chem. 2006, 49, 329-333.
(43) Venkateswarlu, S.; Panchagnula, G. K.; Subbaraju, G. V. Synthesis and Antioxidative Activity of 3′,4′,6,7-Tetrahydroxyaurone, a Metabolite of Bidens frondosa. Biosci. Biotechnol. Biochem. 2004, 68, 2183-2185.
(44) Ashok, D.; Rangu, K.; Gundu, S.; Lakkadi, A.; Tigulla, P. Microwave-Assisted Synthesis, Molecular Docking, and Biological Evaluation of 2-Arylidene-2H-furo[2,3-f]chromen-3(7H)-ones as Antioxidant and Antimicrobial Agents. Med. Chem. Res. 2017, 26, 1735-1746. Campanico, A.; Carrasco, M. P.; Njoroge, M.; Seldon, R.; Chibale, K.; Perdigao, J.; Portugal, I.; Warner, D. F.; Moreira, R.; Lopes, F. Azaaurones as Potent Antimycobacterial Agents Active against MDR- and XDR-TB. Chem. Med. Chem. 2019, 14, 1537-1546. Jardosh, H. H.; Patel, M. P. Antimicrobial and Antioxidant Evaluation of New Quinolone Based Aurone Analogs. Arab. J. Chem. 2017, 10, S3781-S3791. Kumar, G.; Lathwal, E.; Saroha, B.; Kumar, S.; Kumar, S.; Chauhan, N. S.; Kumar, T. Synthesis and Biological Evaluation of Quinoline-Based Novel Aurones. Chem. Select 2020, 5, 3539-3543. Tiwari, K. N.; Monserrat, J.-P.; Hequet, A.; Ganem-Elbaz, C.; Cresteil, T.; Jaouen, G.; Vessières, A.; Hillard, E. A.; Jolivalt, C. In vitro Inhibitory Properties of FerroceneSubstituted Chalcones and Aurones on Bacterial and Human Cell Cultures. Dalton Trans. 2012, 41, 6451-6457.
(45) Balsera, B.; Mulet, J.; Fernández-Carvajal, A.; Torre-Martínez, R. d. I.; Ferrer-Montiel, A.; Hernández-Jiménez, J. G.; Estévez-Herrera, J.; Borges, R.; Freitas, A. E.; López, M. G.; García-López, M. T.; González-Muñiz, R.; Pérez de Vega, M. J.; Valor, L. M.; Svobodová, L.; Sala, S.; Sala, F.; Criado, M. Chalcones as Positive Allosteric Modulators of α7 Nicotinic Acetylcholine Receptors: A New Target for a Privileged Structure. Eur. J. Med. Chem. 2014, 86, 724-739.
(46) Stieger, N.; Liebenberg, W.; Wessels, J. C. UV Spectrophotometric Method for the Identification and Solubility Determination of Nevirapine. Pharmazie 2009, 64, 690-691.
(47) Mota, F. L.; Queimada, A. J.; Pinho, S. P.; Macedo, E. A. Aqueous Solubility of Some Natural Phenolic Compounds. Ind. & Engg. Chem. Res. 2008, 47, 5182-5189.
(48) Lemos, J. A.; Abranches, J.; Koo, H.; Marquis, R. E.; Burne, R. A. Protocols to Study the Physiology of Oral Biofilms. Methods Mol. Biol. 2010, 666, 87-102.
(49) Koo, H.; Falsetta, M. L.; Klein, M. I. The Exopolysaccharide Matrix: A Virulence Determinant of Cariogenic Biofilm. J. Dent. Res. 2013, 92, 1065-1073. Xiao, J.; Klein, M. I.; Falsetta, M. L.; Lu, B.; Delahunty, C. M.; Yates, J. R., 3rd; Heydorn, A.; Koo, H. The Exopolysaccharide Matrix Modulates the Interaction Between 3D Architecture and Virulence of a Mixed-Species Oral Biofilm. PLoS Pathog. 2012, 8, e1002623.
(50) Koo, H.; Xiao, J.; Klein, M. I.; Jeon, J. G. Exopolysaccharides Produced by Streptococcus mutans Glucosyltransferases Modulate the Establishment of Microcolonies Within Multispecies Biofilms. J. Bacteriol. 2010, 192, 3024-3032.
(51) Bowen, W. H.; Koo, H. Biology of Streptococcus mutans Derived Glucosyltransferases: Role in Extracellular Matrix Formation of Cariogenic Biofilms. Caries Res. 2011, 45, 69-86.
(52) Huffines, J. T.; Scoffield, J. A. Disruption of Streptococcus mutans and Candida albicans Synergy by a Commensal Streptococcus. Sci. Rep. 2020, 10, 19661. Huffines, J. T.; Stoner, S. N.; Baty, J. J.; Scoffield, J. A. Nitrite Triggers Reprogramming of the Oral Polymicrobial Metabolome by a Commensal Streptococcus. Front. Cell Infect. Microbiol. 2022, 12, 833339.
(53) Kabsch, W. XDS. Acta Crystallogr. D. 2010, 66, 125-132. Kabsch, W. Integration, Scaling, Space-Group Assignment and Post-Refinement. Acta Crystallogr. D Biol. Crystallogr. 2010, 66, 133-144.
(54) Evans, P. R. An Introduction to Data Reduction: Space-Group Determination, Scaling and Intensity Statistics. Acta Crystallogr. D Biol. Crystallogr. 2011, 67, 282-292.
(55) Winn, M. D.; Ballard, C. C.; Cowtan, K. D.; Dodson, E. J.; Emsley, P.; Evans, P. R.; Keegan, R. M.; Krissinel, E. B.; Leslie, A. G.; McCoy, A.; McNicholas, S. J.; Murshudov, G. N.; Pannu, N. S.; Potterton, E. A.; Powell, H. R.; Read, R. J.; Vagin, A.; Wilson, K. S. Overview of the CCP4 Suite and Current Developments. Acta Crystallogr. D Biol. Crystallogr. 2011, 67, 235-242.
(56) Murshudov, G. N.; Vagin, A. A.; Dodson, E. J. Refinement of Macromolecular Structures by the Maximum-Likelihood Method. Acta Crystallogr. D Biol. Crystallogr. 1997, 53, 240-255.
(57) Afonine, P. V.; Grosse-Kunstleve, R. W.; Echols, N.; Headd, J. J.; Moriarty, N. W.; Mustyakimov, M.; Terwilliger, T. C.; Urzhumtsev, A.; Zwart, P. H.; Adams, P. D. Towards Automated Crystallographic Structure Refinement with Phenix.refine. Acta Crystallogr. D Biol. Crystallogr. 2012, 68, 352-367. Liebschner, D.; Afonine, P. V.; Baker, M. L.; Bunkóczi, G.; Chen, V. B.; Croll, T. I.; Hintze, B.; Hung, L. W.; Jain, S.; McCoy, A. J.; Moriarty, N. W.; Oeffner, R. D.; Poon, B. K.; Prisant, M. G.; Read, R. J.; Richardson, J. S.; Richardson, D. C.; Sammito, M. D.; Sobolev, O. V.; Stockwell, D. H.; Terwilliger, T. C.; Urzhumtsev, A. G.; Videau, L. L.; Williams, C. J.; Adams, P. D. Macromolecular Structure Determination Using X-rays, Neutrons and Electrons: Recent Developments in Phenix. Acta Crystallogr. D Struct. Biol. 2019, 75, 861-877.
(58) Emsley, P.; Lohkamp, B.; Scott, W. G.; Cowtan, K. Features and Development of Coot. Acta Crystallogr. D Biol. Crystallogr. 2010, 66, 486-501.
(59) Davies, G. J.; Wilson, K. S.; Henrissat, B. Nomenclature for Sugar-Binding Subsites in Glycosyl Hydrolases. Biochem. J. 1997, 321 (Pt 2), 557-559.
(60) Zhang, Q.; Ma, Q.; Wang, Y.; Wu, H.; Zou, J. Molecular Mechanisms of Inhibiting Glucosyltransferases for Biofilm Formation in Streptococcus mutans. Int. J. Oral Sci. 2021, 13, 30.
(61) Cusack, K. P.; Arnold, L. D.; Barberis, C. E.; Chen, H.; Ericsson, A. M.; Gaza-Bulseco, G. S.; Gordon, T. D.; Grinnell, C. M.; Harsch, A.; Pellegrini, M.; Tarcsa, E. A ¹³C NMR Approach to Categorizing Potential Limitations of α,β-Unsaturated Carbonyl Systems in Drug-Like Molecules. Bioorg. Med. Chem. Lett. 2004, 14, 5503-5507.
(62) McGovern, S. L.; Helfand, B. T.; Feng, B.; Shoichet, B. K. A Specific Mechanism of Nonspecific Inhibition. J. Med. Chem. 2003, 46, 4265-4272.
(63) Kozlovskaya, V.; Chen, J.; Tedjo, C.; Liang, X.; Campos-Gomez, J.; Oh, J.; Saeed, M.; Lungu, C. T.; Kharlampieva, E. pH-Responsive Hydrogel Cubes for Release of Doxorubicin in Cancer Cells. J. Mater. Chem. B. 2014, 2, 2494-2507.
(64) Xue, B.; Kozlovskaya, V.; Liu, F.; Chen, J.; Williams, J. F.; Campos-Gomez, J.; Saeed, M.; Kharlampieva, E. Intracellular Degradable Hydrogel Cubes and Spheres for Anti-Cancer Drug Delivery. ACS Appl. Mater. Interfac. 2015, 7, 13633-13644.
(65) Xue, B.; Wang, W.; Qin, J. J.; Nijampatnam, B.; Murugesan, S.; Kozlovskaya, V.; Zhang, R.; Velu, S. E.; Kharlampieva, E. Highly Efficient Delivery of Potent Anticancer Iminoquinone Derivative by Multilayer Hydrogel Cubes. Acta Biomater. 2017, 58, 386-398.
(66) Liu, P.; Luo, Q.; Guan, Y.; Zhang, Y. Drug Release Kinetics from Monolayer Films of Glucose-Sensitive Microgel. Polymer 2010, 51, 2668-2675. Sun, S.; Wu, P. A One-Step Strategy for Thermal- and pH-Responsive Graphene Oxide Interpenetrating Polymer Hydrogel Networks. J. Mater. Chem. B. 2011, 21, 4095-4097.
(67) Hazlett, K. R.; Michalek, S. M.; Banas, J. A. Inactivation of the gbpA Gene of Streptococcus mutans Increases virulence and Promotes In Vivo Accumulation of Recombinations Between the Glucosyltransferase B and C Genes. Infect. Immun. 1998, 66, 2180-2185. Michalek, S. M.; McGhee, J. R.; Shiota, T.; Devenyns, D. Virulence of Streptococcus mutans: Cariogenicity of S. mutans in Adult Gnotobiotic Rats. Infect. Immun. 1977, 15, 466-471.
(68) Keyes, P. H. Dental Caries in the Molar Teeth of Rats. II. A Method for Diagnosing and Scoring Several Types of Lesions Simultaneously. J. Dent. Res. 1958, 37, 1088-1099.
(69) Liu, C.; Worthington, R. J.; Melander, C.; Wu, H. A New Small Molecule Specifically Inhibits the Cariogenic Bacterium Streptococcus mutans in Multispecies Biofilms. Antimicrob. Agents Chemother. 2011, 55, 2679-2687.
(70) Zhang, Q.; Nguyen, T.; McMichael, M.; Velu, S. E.; Zou, J.; Zhou, X.; Wu, H. New Small-Molecule Inhibitors of Dihydrofolate Reductase Inhibit Streptococcus mutans. Int. J. Antimicrob. Agents 2015, 46, 174-182.
(71) Lynch, D. J.; Michalek, S. M.; Zhu, M.; Drake, D.; Qian, F.; Banas, J. A. Cariogenicity of Streptococcus mutans Glucan-Binding Protein Deletion Mutants. Oral Health Dent. Manag. 2013, 12, 191-199. Peng, X.; Zhang, Y.; Bai, G.; Zhou, X.; Wu, H. Cyclic di-AMP mediates biofilm formation. Mol. Microbiol. 2016, 99, 945-959.
(72) Mieher, J. L.; Schormann, N.; Patel, M.; Wu, H.; Deivanayagam, C. Cloning, Expression, Purification, and Preliminary Characterization of Single-Crystal X-Ray Diffraction of Glucosyltransferase B of Streptococcus mutans. N. Prod. Commun. 2019, 14, 5.
(73) McCoy, A. J.; Grosse-Kunstleve, R. W.; Adams, P. D.; Winn, M. D.; Storoni, L. C.; Read, R. J. Phaser crystallographic software. J. Appl. Crystallogr. 2007, 40, 658-674.
(74) Kovalevskiy, O.; Nicholls, R. A.; Long, F.; Carlon, A.; Murshudov, G. N. Overview of refinement procedures within REFMAC5: utilizing data from different sources. Acta Crystallogr. D Struct. Biol. 2018, 74, 215-227. Murshudov, G. N.; Skubak, P.; Lebedev, A. A.; Pannu, N. S.; Steiner, R. A.; Nicholls, R. A.; Winn, M. D.; Long, F.; Vagin, A. A. REFMAC5 for the refinement of macromolecular crystal structures. Acta Crystallogr. D Biol. Crystallogr. 2011, 67, 355-367. Murshudov, G. N.; Vagin, A. A.; Dodson, E. J. Refinement of Macromolecular Structures by the Maximum-Likelihood Method. Acta Crystallogr. D Biol. Crystallogr. 1997, 53, 240-255.
(75) Emsley, P.; Cowtan, K. Coot: model-building tools for molecular graphics. Acta Crystallogr. D Biol. Crystallogr. 2004, 60, 2126-2132. Emsley, P.; Lohkamp, B.; Scott, W. G.; Cowtan, K. Features and Development of Coot. Acta Crystallogr. D Biol. Crystallogr. 2010, 66, 486-501.

### Example 2

This study focuses on the development of a novel drug delivery platform for the pH-responsive delivery of biofilm inhibitors as a potential avenue to prevent and treat dental caries. Streptococcus mutans is the primary etiological agent for dental caries. The potential of two previously reported biofilm inhibitors for HA5 and HA6 to be therapeutics that have species-specific selectivity towards S. mutans and the ability to preserve the oral microbiome is demonstrated by characterizing the rat oral microbiome in a S. mutans induced dental caries model. Inhibitors HA5 and HA6 were encapsulated into pH-responsive block copolymer vesicles (polymersomes) to generate polymersome-encapsulated biofilm inhibitors, PEHA5 and PEHA6, respectively and their biofilm and growth inhibitory activities against S. mutans and representative strains of oral commensal streptococci have been assessed. The in vivo drug release and antivirulence activities of PEHA5 have been demonstrated in a S. mutans induced rat caries model. A 4-week treatment of S. mutans UA159 infected gnotobiotic rats with 100 μM of PEHA5 resulted in significant reductions in buccal, sulcal, and proximal caries scores compared to an untreated control group without affecting bacterial colonization significantly. The results were comparable to HA5 treatment alone. Taken together, our data suggests that the biofilm-selective therapy using the polymersome-encapsulated biofilm inhibitors is a viable approach for the prevention and treatment of dental caries while preserving the oral microbiome.

Dental caries, commonly known as tooth decay, is a ubiquitous bacterial infectious disease that causes demineralization of enamel and dentin^{[1]}. A recent Lancet study of global burden of 328 major diseases recognizes dental caries as the most prevalent disease worldwide^{[2]}. Although dental plaque contains more than 700 bacterial species living in complex bacterial communities called biofilms, the gram-positive bacterium Streptococcus mutans, characterized by its ability to form tenacious biofilms is considered to be the primary etiological agent for this disease^{[3, 4]}. Biofilm formation is initiated by the attachment of commensal streptococci such as Streptococcus sanguinis and Streptococcus gordonii to the tooth surface and the subsequent intra- and inter-species microbial interactions^{[5-7]}. Low numbers of cariogenic bacteria often live together with their benign commensal counterparts in the oral cavity as multispecies biofilm communities^{[8-10]}. Under the disease conditions, pathogenic bacteria overgrow the commensals disturbing the delicate balance between them^{[11, 12]}. Current antimicrobial treatments for dental caries such as oral rinses affect both pathogenic and commensal bacteria alike. Therefore, it would be beneficial if the studies aimed at developing new caries treatments focus on identifying antibiofilm agents that have no adverse impact on the growth of oral commensal species. One of the strategies to accomplish this goal is by developing inhibitors of S. mutans virulence factors such as extracellular glucosyl transferases (Gtfs)^{[13]}. S. mutans secreted enzymes GtfB and GtfC are primarily responsible for the synthesis of water-insoluble glucans^{[14, 15]} and GtfD is responsible for the synthesis of water-soluble glucans^{[16, 17]}. Functions of S. mutans Gtfs are essential for the glucan synthesis, biofilm formation and the resulting cariogenesis Therefore, inhibiting S. mutans Gtfs is an excellent strategy to specifically inhibit its biofilm formation without affecting its viability and the viability of oral commensal bacterial species. Two such S. mutans Gtf inhibitors reported from our lab are compounds HA5 and HA6^{[18]}.

The goal of the present study is to encapsulate HA5 or HA6 in pH-responsive polymer nanoplatforms and explore their on-demand pH-responsive delivery in the oral cavity to prevent or treat dental caries. The pH-responsive delivery of antibacterial agents is a highly desirable approach to treat dental caries as the pH level in oral cavity is one of the critical factors contributing to the demineralization process of tooth enamel. The human salivary system maintains a healthy non-harmful pH of 6.0-7.5 in the oral cavity^{[19, 20]} under physiological conditions controlled by three buffer systems: 1) bicarbonate, 2) phosphate and 3) salivary proteins^{[21, 22]}. Under pathogenic oral conditions, biofilm ferments the dietary carbohydrates to produce acidic byproducts such as lactic acid leading to a drop in salivary pH to less than 5.5, which is harmful to the tooth enamel and dentin^{[21-24]}. Therefore, dental caries treatment would tremendously benefit from an antibiofilm agent that is delivered on the tooth surface in a pH-responsive manner.

Given the challenges of poor solubility of small molecule antibacterial agents, difficulty of penetration into biofilms, and lack of retention of the drugs within biofilm, the use of nanomaterials for the localized delivery of antibacterial agents is a prudent approach to treat dental caries^{[25-29]}. Examples of such studies are the delivery of farnesol and myricetin using nanoparticle carriers to inhibit biofilm^{[30]}, delivery of farnesol using pH-responsive micelles (PPi-Far-PM) and the use of nano systems such as mesoporous silica nanoparticle (MSN)^{[32, 33]}, liposome^{[34]}, halloysite nanotube (HNT)^{[35]}, and polyamidoamine (PAMAM)^{[36]} for controlled release of anticaries drugs. However, none of these approaches have been translated to clinical use so far as their in vivo efficacies are either modest or unproven.

Our interest is focused on developing a novel polymersome drug delivery system with built-in pH-sensitivity for the delivery of biofilm inhibitors as a potential caries treatment. Polymersomes are hollow polymeric spheres with an aqueous core and a polymer membrane that has close similarity to the membrane of liposomes^{[37, 38]}. Polymersomes are ideal delivery platforms for small-molecule biofilm inhibitors as their amphiphilicity makes them capable of encapsulating both hydrophilic and hydrophobic molecules in their core and polymer shell, respectively^{[39]}. Polymersomes are mechanically robust with efficient drug loading capacity and ability to respond to environmental stimuli such as pH or temperature^{[40-42]}. Such polymer vesicles made from block copolymers of polybutadiene-b-poly(L-glutamic acid) and polyethyloxide-β-polycaprolactam have been shown to release their cargo through the vesicle disassembly due to the presence of the degradable bonds within their structure^{[42]}. We specifically designed spherical block copolymer vesicles to encapsulate our less water-soluble biofilm inhibitors. These hollow vesicles were self-assembled from poly(N-vinylpyrrolidone)₈-block-poly(dimethylsiloxane)₆₄-block-poly(N-vinyl-pyrrolidone)₈(PVPON₈-PDMS₆₄-PVPON₈) block copolymer into ~30-nm spherical hollow nanovesicles via a nanoprecipitation method. The synthesis was carried out by the reversible addition-fragmentation chain transfer (RAFT) polymerization we have reported previously^{[43]}. Due to the presence of acid-labile ester (—COO—) linkages between PDMS and PVPON blocks, the assembled polymersome vesicles are degraded at pH<5 and release the cargo^{[43]}.

### Results and Discussion

Biofilm inhibitors HA5 and HA6 are excellent candidates for polymersome encapsulation as they inhibited S. mutans biofilm with IC₅₀ values of 6.42 μM and 18.92 μM, respectively without affecting the growth of commensal species S. gordonii and S. sanguinis at their biofilm inhibiting doses^{[18]}. They were also found to have solubilities of 120.09 μg/mL and 90.77 μg/mL, suitable for the encapsulation into polymersome vesicles^{[18]}. Both inhibitors HA5 and HA6 were synthesized in large scale using the synthetic protocols reported from our lab recently^{[18]}.

Detailed evaluation of Gtf inhibition, biofilm inhibition and growth inhibition activities of compound HA5 has already been reported in our prior publication^{[18]}. Additional in vitro and in vivo evaluations were conducted for compound HA6 to ensure its suitability for encapsulation as described in the following sections.

### Biofilm and Growth Inhibition by HA6

Compound HA6 inhibited S. mutans biofilm in a dose dependent manner with an IC₅₀ value of 18.92±0.39 μM (FIG. 2.2A). Staining of bacterial cells within biofilms with Syto-9 showed significant reduction in biofilms at 15 μM and a complete inhibition at 30 μM of HA6 (FIG. 2.2E-I). The presence of glucans, which were stained with Cascade Blue-dextran conjugated dye, was significantly reduced at 15 μM and no glucan formation was evident at 30 μM of HA6 (FIG. 2.2E-II). In addition, propidium iodide was used to determine the presence of extracellular DNA (eDNA) in S. mutans biofilms. Again, there was a noticeable reduction of eDNA at 15 μM and almost complete absence of eDNA at 30 μM of HA6 (FIG. 2.2E-III). These findings reaffirm that compound HA6 inhibited S. mutans biofilms by preventing the synthesis of glucans and minimizing the presence of eDNA, two integral biofilm matrix elements crucial for S. mutans biofilm formation.

To determine if HA6 only selectively inhibits S. mutans biofilms without affecting the planktonic growth of S. mutans and oral commensal species, the effects of HA6 on the viability of two representative oral commensal streptococci, S. gordonii and S. sanguinis, along with S. mutans at 25 μM and 50 μM doses were evaluated. As shown in FIG. 2.2B, compound HA6 did not inhibit the growth of S. sanguinis, while it showed about 10% inhibition of the growth of S. gordonii compared to the control groups at these doses that are much higher than its biofilm IC₅₀ value of 18.92 μM. Similarly, the compound HA6 did not inhibit S. mutans viability at these doses (FIG. 2.2B). In addition, we have demonstrated that HA6 did not significantly reduce the biofilms of the commensal species S. gordonii and S. Sanguinis at 25 μM (FIG. 2.2C-D).

### Effect of HA5 of HA6 on Rat Oral Microbiome.

Although HA5 and HA6 are efficient inhibitors of S. mutans biofilm and cariogenic activity^{[18]}, it is important to ensure that these compounds do not have any deleterious impact on the healthy oral microbial community. To understand how these compounds affect the residential bacterial community, we conducted an in vivo evaluation of both HA5 and HA6 and compared to the untreated and NaF-treated rats using a well-established gnotobiotic rat model of dental caries^{[50-54]}. Treatment groups used in this study were inhibitor HA5 (100 μM), inhibitor HA6 (100 μM), and vehicle. A UA159 infection only group served as a negative control and a NaF (250 ppm) served as a positive control.

All rats from the experimental groups and from the control groups were colonized with S. mutans UA159. A 4-week treatment of infected gnotobiotic rats with HA5 or HA6 resulted in significant reductions in buccal caries scores from the enamel (E) and dentinal slight (Ds), dentinal medium (Dm), and dentinal extensive (Dx) compared to control groups (Table 1). Similar reductions were observed in sulcal and proximal caries scores (Table 2). The effect of the treatment on dentinal moderate (Dm) and dentinal extensive (Dx) scores in the proximal area were not recorded as there were no observable caries lesions in both control and the treated groups. The observed reductions in caries scores by HA5 or HA6 were slightly lower than the positive control NaF treatment. However, it should be noted that the concentration of NaF (250 ppm=5.95 mM) is about 59-fold higher than the inhibitor treatment dose of 100 μM. In addition, the rats treated with the compound HA5 or HA6 did not experience significant weight loss over the course of the study, suggesting that the compounds are non-toxic (Table 3). Furthermore, HA5 or HA6 treatment did not affect bacterial colonization significantly compared to control group (Table 3).

TABLE 1
Effect of HA5 or HA6 treatment on S. mutans
UA159 induced buccal caries.

| | Buccal Mean | | | |
|---|---|---|---|---|
| | Caries Scores | | | |
| Treatment | (±SEM) | | | |
| Group | E | Ds | Dm | Dx |
| UA 159 infected and | 13.2 ± 0.4 | 9.2 ± 0.6 | 6.2 ± 0.7 | 3.6 ± 0.4 |
| untreated | | | | |
| HA5 treated (100 μM) | 7.8 ± 0.4 | 6.6 ± 0.5 | 3.6 ± 0.4 | 2.2 ± 0.7 |
| HA6 treated (100 μM) | 8.2 ± 0.7 | 6.8 ± 0.5 | 4.0 ± 0.7 | 1.2 ± 0.5 |
| NaF treated (250 ppm) | 6.2 ± 0.9 | 3.2 ± 0.9 | 1.6 ± 0.5 | 0.4 ± 0.2 |

Enamel (E); Dentinal slight (Ds); Dentinal moderate (Dm); Dentinal extensive (Dx), n = 5.

TABLE 2
Effect of HA5 or HA6 treatment on S. mutans UA159 induced sulcal and proximal
caries.

| | Mean Caries Scores (± SEM) | | | | Proximal Mean | |
|---|---|---|---|---|---|---|
| | Sulcal Mean Caries Scores | | | | Caries Scores | |
| Treatment | (± SEM) | | | | (± SEM) | |
| Group | E | Ds | Dm | Dx | E | Ds |
| UA159 infected and untreated | 25.8 ± 1.2 | 18.8 ± 1.3 | 12.8 ± 0.7 | 6.6 ± 0.3 | 8.0 ± 0.0 | 5.8 ± 0.7 |
| HA5 treated (100 μM) | 16.4 ± 2.2 | 11.2 ± 0.6 | 4.6 ± 0.2 | 1.0 ± 0.3 | 1.0 ± 0.3 | 0.0 ± 0.0 |
| HA6 treated (100 μM) | 14.6 ± 0.9 | 11.2 ± 0.7 | 6.4 ± 0.4 | 1.6 ± 0.4 | 1.2 ± 0.8 | 0.0 ± 0.0 |
| NaF treated (250 ppm) | 15.2 ± 0.7 | 10.4 ± 0.5 | 5.4 ± 0.4 | 1.6 ± 0.5 | 0.0 ± 0.0 | 0.0 ± 0.0 |

Enamel (E); Dentinal slight (Ds); Dentinal moderate (Dm); Dentinal extensive (Dx), n = 5.

TABLE 3
Effect of HA5 or HA6 treatment on S. mutans UA159 CFU
and the body weight of the treated animals.

| | | | Animals | |
|---|---|---|---|---|
| Treatment | CFU/mL (×10⁵) | | Weight | |
| Group | MS | BAP | (g) | Number |
| UA 159 infected and | 2.3 ± 1.2 | 3.3 ± 2.0 | 161 ± 12 | 5 |
| untreated | | | | |
| HA5 treated (100 μM) | 2.2 ± 0.8 | 3.9 ± 1.4 | 156 ± 16 | 5 |
| HA6 treated (100 μM) | 1.3 ± 0.4 | 1.9 ± 0.5 | 165 ± 13 | 5 |
| NaF treated (250 ppm) | 1.6 ± 0.6 | 2.8 ± 0.7 | 145 ± 12 | 5 |

Mitis-Salivarius agar (MS); Blood agar plate (BAP)

During this study, rat oral microbiome samples were collected from individual rats (n=5) at the following time points: before the experiment (Native), after inoculation of S. mutans and the start of a caries-promoting diet (Sm+CPD), after two weeks of treatment with the compound (2-week), and at the end of the study (END). The microbiota between the groups at different time points was analyzed for oral bacterial composition and abundance. Oral swabs collected before and after interventions with these compounds were analyzed using the 16s rRNA gene sequencing method. Both ‘within’ (alpha diversity) and ‘between’ (beta diversity) sample diversities were calculated over time for each treatment group using MicrobiomeAnalyst 2.0^{[55]}.

The major phyla detected in the study are Firmicutes, Proteobacteria, Bacteroides, Actinobacteria, Verrucomicrobia, Epsilonbacteraeota, Tenericutes, Cyanobacteria, and Spirochaetes. Phylum level comparison of oral microbiome samples from initial infection of S. mutans after 2-week of treatment and 4-week treatment showed that the phylum Firmicutes dominated the native microbiome and to a lesser extent by phyla Proteobacteria and Bacteroides. Treatment with the compounds HA5 or HA6 did not perturb the overall rat oral microbiome at phylum levels significantly (FIG. 2.3A). Each color represents 1 phylum, and the length of the bar reflects relative abundance. The results were similar to the NaF treatment and the control: infected untreated animals.

The family level comparison within the major phylum, Firmicutes from the initial infection of S. mutans to after 4-week of treatment was carried out. The major families detected within the phylum Firmicutes throughout the study are Lachnospiraceae, Ruminococcaceae, Lactobacillus, Erysipelotrichaceae, and Streptococcaceae. Each color represents 1 family, and the length of bar reflects relative abundance (FIG. 2.3B). The results suggested that the Lachnospiraceae family dominated in the native microbiome and, to a lesser extent by families Ruminococcaceae and Lactobacilloae and the treatment with compounds HA5 or HA6 did not perturb the overall rat oral microbiome significantly within this phylum (FIG. 2.3B). However, an increase in the abundance of the Streptococcaceae family was observed during the second week of the study for untreated and fluoride treated groups. This increase was not sustained till the end of the study as the week 4 abundance of Streptococcaceae composition was found to be similar to the native group suggesting that the observed increase in abundance during week 2 was likely an experimental artifact. Overall, the results of compound treatment were similar to NaF treatment and the control: infected untreated animals.

The principal component analysis plots of alpha-diversity of all animals in groups over time: Native (black) and END (gray) are presented in FIG. 2.3C. As in untreated control groups, the compounds treated groups also maintained an increasing trend in the alpha diversity at the end of the treatment. Both compounds HA5 and HA6 maintained the beta diversity without any significant deviations (PERMANOVA, P-value>0.05) from the native community (FIG. 2.3D). None of the groups showed any change in the community after Sm+CPD treatment, except the NaF-treated group (PERMANOVA, P-value>0.001), where the community was shifted, and the samples were clustered separately. However, after 2 weeks of treatment, it shifted closer to the native state. Similar to what we observed in the untreated group, the intervention for a total of 4 weeks with these compounds did not show any shift in the oral bacterial community, indicating the harmless nature of these compounds towards other commensal bacteria in the oral cavity.

Killing and elimination of S. mutans by synthetic antimicrobial peptide C16G2 in an in vitro oral biofilm model in saliva also nonspecifically eliminated noncariogenic species, leading to a drastic shift of the structure of the microbiota^{[56]}. A novel small molecule 3F1 significantly reduced caries scores in vivo without affecting the rat oral microbiome^{[57]}. Our microbiome analysis data suggests that the novel small molecules HA5 and HA6 can significantly reduce the caries scores in vivo without affecting the rat oral microbiome similar to 3F1, and the inhibition of S. mutans biofilm is sufficient to decrease the incidence of dental caries. Any type of dysbiosis in the oral microbiota may favor the dental caries promoting organisms and result in adverse effects. Thus, targeting the bacterial species that promote dental carries without any major perturbation to normal healthy microbiota has greater implications in maintaining dental health.

Design of pH-Responsive Polymersome Vesicles.

Spherical block copolymer vesicles that allow the encapsulation of both hydrophilic and hydrophobic drugs were designed and synthesized. The hollow block copolymer vesicles were assembled from PVPON₈-PDMS₆₄-PVPON; block copolymer into ~30 nm vesicles using a nanoprecipitation method^{[58, 59]}. The block-copolymer was synthesized by reversible addition-fragmentation chain transfer (RAFT) polymerization of N-vinylpyrrolidone (VPON) from PDMS as we have previously reported (FIG. 2.4A)^{[60]}. In this protocol, bis(hydroxyalkyl) poly (dimethylsiloxane) (PDMS₆₄) was first modified with 2-bromopropionyl bromide followed by potassium ethyl xanthogenate resulting in the PDMS macro-initiator. PVPON₈-PDMS₆₄-PVPON: was synthesized by RAFT polymerization of N-vinylpyrrolidone (VPON) monomer by controlling the reaction time with a feed ratio of 1:1:95 by weight of PDMS-initiator/AIBN/monomer. Monomer conversion and the molecular weight of the purified copolymer were determined by 1H-NMR (FIG. 2.4B) and was found to be Mn=7,070 g/mol.

Due to the presence of acid labile ester linkages between PDMS and PVPON blocks in the block-copolymer, the assembled polymersome vesicles are degraded when pH is lowered to <5. FIG. 2.4D shows that turbidity of the empty vesicle solution (2 mL, 0.5 mg/mL) incubated in 0.01 M phosphate buffer at pH=3 at 37° C. for 24 h decreased two times as measured by fluorescence spectroscopy (Varian, Cary Eclipse) at λ=700 nm. The images of the vesicle solution before and after the pH exposure demonstrate that the lowering of pH resulted in the vesicle degradation as indicated by the transparent solution (FIG. 2.4C).

### Polymersome Encapsulation of Compounds HA5 or HA6.

The PVPON-b-PDMS-b-PVPON triblock-copolymer solution in ethanol (5.0 mg/mL; 1 mL) was added dropwise to 4.0 mL of HA5 (0.045 mg/mL), or HA6 (0.035 mg/mL), solution in DI water at room temperature and left stirring for 2 h. Then, the obtained solution was dialysed in DI water for 48 h using a Float-a-Lyzer (MWCO 1000 Da, Fisher Scientific) to remove ethanol, followed by dialysis in DI water for 72 h using a Float-a-Lyzer (MWCO 100 kDa, Fisher Scientific) to remove an excess of the drug. The hydrodynamic sizes of purified PEHA5 and PEHA6 were measured using a Nano-ZS Zetasizer (Malvern Pananalytical) equipped with a He—Ne laser (663 nm) at 25° C. The average hydrodynamic diameters were measured to be 33±10 nm for empty vesicles, and 33±11 nm and 28±9 nm for PEHA5 and PEHA6, respectively (FIG. 2.4E). The concentration of encapsulated drug was calculated using a UV-visible spectroscopy using calibration curves for HA5 (λₘₐₓ=444 nm) and HA6 (λₘₐₓ=412 nm) (FIG. 2.4F).

### Biofilm and Growth Inhibitory Activities of PEHA5.

Prior to in vivo evaluations, the biofilm inhibitory activities of PEHA5 were investigated. PEHA5 inhibited S. mutans biofilm in a dose-dependent manner with an IC₅₀ value of 12.04±1.51 μM (FIG. 2.5A). Staining of the bacterial cells within biofilms with Syto-9 showed significant reduction in biofilms at 10 μM and a complete inhibition at 20 μM of PEHA5 (FIG. 2.5E-I). The presence of glucans, which were stained with Cascade Blue-dextran conjugated dye, was significantly reduced at 10 μM and no glucan formation was evident at 20 μM of PEHA5 (FIG. 2.5E-II). In addition, propidium iodide was used to determine the presence of eDNA in S. mutans biofilms. Again, there was a noticeable reduction of eDNA at 10 μM and almost complete absence of eDNA at 20 μM of PEHA5 (FIG. 2.5E-III). These findings reaffirm that PEHA5 exhibited S. mutans biofilm inhibitory activities similar to what is reported for unencapsulated compound HA5^{[18]}. The effects of unloaded polymersome vesicles on S. mutans biofilm were compared with the control (1% DMSO) and the biofilm inhibitory activities of PEHA5 and HA5 at a single dose 50 μM concentration. Clearly, empty polymersome vesicles did not inhibit the biofilm as compared to 80% inhibition by PEHA5 and 95% inhibition by HA5 (FIG. 2.5B). The planktonic growth of S. mutans was not affected by PEHA5 at the range of doses of 5 μM-50 μM (FIG. 2.5C). In addition, the effect of PEHA5 on the planktonic growth of two commensal Streptococci, S. gordonii and S. Sanguinis were compared with the control (1% DMSO) and HA5 at a single treatment dose of 50 μM. Compared to control, PEHA5 slightly inhibited the growth of S. gordonii and S. sanguinis at this dose, but the effects were minimal (FIG. 2.5D).

### Biofilm and Growth Inhibitory Activities of PEHA6.

The biofilm inhibitory activities of PEHA6 were investigated. PEHA6 inhibited S. mutans biofilm in a dose-dependent manner with an IC₅₀ value of 8.09±2.92 μM (FIG. 2.6A). Staining of the bacterial cells within biofilms with Syto-9 showed significant reduction in biofilms at 10 μM and a complete inhibition at 20 μM of PEHA6 (FIG. 2.6E-I). The presence of glucans, which were stained with Cascade Blue-dextran conjugated dye, was significantly reduced at 10 μM and no glucan formation was evident at 20 μM of PEHA6 (FIG. 2.6E-II). In addition, propidium iodide was used to determine the presence of eDNA in S. mutans biofilms. Again, there was a substantial decrease in eDNA at 10 μM and almost complete absence of eDNA at 20 M of PEHA6 (FIG. 2.6E-III). These findings suggest that PEHA6 demonstrated S. mutans biofilm inhibitory activities comparable to unencapsulated compound HA6. The effects of PEHA6 on S. mutans biofilm were compared with the control (1% DMSO) and the biofilm inhibitory activities of HA5 and HA6 side by side at a single dose, 50 μM concentration. Clearly, PEHA6 inhibited 95% of the biofilm closely resembling the efficacy of HA6, with HA5 showing an 85% inhibition of biofilm (FIG. 2.6B). The planktonic growth of S. mutans was not affected by PEHA6 at the range of doses of 5 μM-50 μM (FIG. 2.6C). In addition, the effect of PEHA6 on the planktonic growth of S. mutans was compared with HA5, HA6 at 50 μM. Compared to control, PEHA6 and HA6 inhibited the planktonic growth slightly while HA5 did not inhibit the growth significantly at this dose (FIG. 2.6D).

Reduction of S. mutans Virulence In Vivo by PEHA5

The effect of PEHA5 on S. mutans colonization and virulence was compared side-by-side with HA5 using a well-established gnotobiotic rat model of dental caries^{[50-54]}. A (vehicle+infection only) group was included as a negative control. All rats from the experimental groups and from the control group were colonized with S. mutans UA159. A 4-week treatment of S. mutans UA159 infected gnotobiotic rats with 100 μM of PEHA5 or HA5 resulted in significant reductions in buccal caries scores for enamel (E), dentinal slight (Ds), dentinal moderate (Dm), and dentinal extensive (Dx) lesions compared to control groups (Table 4). Similar reductions in caries scores were also observed in sulcal caries scores and proximal caries scores (Table 5). We were unable to record the effect of the treatment on dentinal moderate (Dm) and dentinal extensive (Dx) scores in the proximal area due to the absence of dentinal lesions in the control and treated groups. The observed reductions in caries scores by PEHA5 were comparable with HA5, with the PEHA5 displaying slightly better in vivo activity, possibly due to the slow pH-dependent release of the drug from PEHA5 over the treatment period. In addition, the rats treated with PEHA5 or HA5 did not experience any weight loss over the course of the study in comparison with the control group, suggesting the non-toxic nature of the material and the compound (Table 6). Furthermore, PEHA5 or HA5 treatment did not affect bacterial colonization significantly (Table 6). Overall, our data suggests that PEHA5 releases HA5 under the acidic conditions of the dental caries infected oral cavity and reduce the cariogenic activity. The reductions in caries scores produced by PEHA5 is comparable to that achieved by HA5 treatment alone.

TABLE 4
Effect of PEHA5 or HA5 treatment on S. mutans UA159
induced buccal caries.

| | Buccal Mean | | | |
|---|---|---|---|---|
| | Caries Scores | | | |
| Treatment | (±SEM) | | | |
| Group | E | Ds | Dm | Dx |
| UA 159 infected and | 13.8 ± 0.9 | 11.4 ± 0.5 | 8.2 ± 0.4 | 5.6 ± 0.7 |
| untreated | | | | |
| PEHA5 treated (100 μM) | 7.4 ± 0.6 | 5.4 ± 0.7 | 4.0 ± 0.6 | 1.4 ± 0.9 |
| HA5 treated (100 μM) | 9.0 ± 0.6 | 6.4 ± 1.0 | 2.8 ± 0.5 | 1.4 ± 0.4 |

Enamel (E); Dentinal slight (Ds); Dentinal moderate (Dm); Dentinal extensive (Dx)

TABLE 5
Effect of PEHA5 or HA5 treatment on S. mutans UA159 induced sulcal and
proximal caries.

| | Mean Caries Scores (± SEM) | | | | Proximal Mean | |
|---|---|---|---|---|---|---|
| | Sulcal Mean Caries Scores | | | | Caries Scores | |
| Treatment | (± SEM) | | | | (± SEM) | |
| Group | E | Ds | Dm | Dx | E | Ds |
| UA159 infected and untreated | 24.2 ± 0.9 | 18.4 ± 0.7 | 13.4 ± 0.9 | 7.0 ± 0.3 | 6.8 ± 0.8 | 4.6 ± 1.0 |
| PEHA5 treated (100 μM) | 16.0 ± 0.8 | 12.6 ± 0.7 | 6.6 ± 0.5 | 2.6 ± 0.5 | 4.0 ± 1.1 | 1.6 ± 1.0 |
| HA5 treated (100 μM) | 20.6 ± 0.8 | 15.0 ± 0.9 | 9.4 ± 0.5 | 4.8 ± 0.9 | 5.2 ± 0.5 | 3.4 ± 0.6 |

Enamel (E); Dentinal slight (Ds); Dentinal moderate (Dm); Dentinal extensive (Dx)

TABLE 6
Effect of PEHA5 or HA5 treatment on S. mutans UA159 CFU
and the body weight of the treated animals.

| | | | Animals | |
|---|---|---|---|---|
| | CFU/mL (×10⁵) | | Weight | Number |
| Group | MS | BAP | (g) | (n) |
| UA159 infected and | 3.2 ± 0.6 | 3.9 ± 0.4 | 140 ± 9 | 5 |
| untreated | | | | |
| PEHA5 treated (100 μM) | 1.4 ± 0.4 | 3.4 ± 0.9 | 149 ± 16 | 5 |
| HA5 treated (100 μM) | 5.5 ± 2.6 | 5.2 ± 1.4 | 145 ± 14 | 5 |

Mitis-Salivarius agar (MS); Blood agar plate (BAP)

### Conclusions

Two previously reported Streptococcus mutans biofilm inhibitors HA5 and HA6 were prepared in larger scale and further evaluated for S. mutans Gtf inhibition, biofilm inhibition and growth inhibition activities against representative strains of oral commensal streptococci. The in vivo antivirulence activities and the potential of HA5 or HA6 to be a therapeutic that combines both species-specific selectivity towards S. mutans and preserves the oral microbiome is demonstrated in vivo by characterizing the oral microbiome in a rat caries model. Phylum and family level comparison of the treatment groups from initial infection of S. mutans to after 2-week and 4-week treatment with HA5 or HA6 showed selective control of S. mutans by the inhibitors without perturbing the overall rat oral microbiome significantly. Previous studies targeting S. mutans biofilm dispersion achieved similar outcomes by reducing caries and preserving overall microbiota, however, the underlying mechanism of the dispersion agent is unknown^{[57]}. Both inhibitors HA5 and HA6 were encapsulated into pH-responsive block copolymer vesicles to generate a polymersome-encapsulated biofilm inhibitors, PEHA5 and PEHA6 respectively and their biofilm and growth inhibitory activities against S. mutans and representative strains of oral commensal Streptococci have been assessed. A 4-week treatment of S. mutans UA159 infected gnotobiotic rats with 100 μM PEHA5 resulted in significant reductions in buccal, sulcal, and proximal dental caries scores compared to untreated control groups. These outcomes were comparable to those observed with 100 μM of HA5 treatment. Taken together, these results suggest that the compound HA5 and the polymersome encapsulated material, PEHA5 selectively targeted S. mutans virulence factors; Gtfs and Gtf-mediated biofilm formation, rather than a simple inhibition of bacterial growth and are very effective in reducing dental caries in vivo. Overall, our data suggests that the S. mutans biofilm-specific therapy using HA5, HA6, or the polymersome encapsulated materials reported here is a viable approach for preventing caries while preserving the oral microbiome.

### EXPERIMENTAL

### General Considerations

All bacterial strains (S. mutans UA159, S. gordonii DL1, and S. sanguinis SK36) were inoculated statically at 37° C. under 5% CO₂ environment in Todd Hewitt Broth (THB) for 24 h^{[61]}. The cultures were then diluted with fresh THB (1:5) and reinoculated until the optical density at 470 nm reaches 1. The optical density was read using BioTek 800 TS absorbance reader at 470 nm for bacterial growth and 562 nm for biofilm stained with crystal violet. Data was plotted in Prism 10.0.2.

### Biofilm Inhibition Assays

Biofilm inhibition assays were performed in polystyrene microtiter 96-well plates. Stock solutions were prepared in chemically defined medium (CDM) with 1% sucrose, 1% bacteria cultures and various concentrations of the small molecule inhibitors to examine their activity against biofilm formation as described^{[62, 63]}. These stocks were assayed in 96 well plates in triplicate and incubated at 37° C. and 5% CO₂ for 16 h. After reading optical density for bacterial growth, the plate was washed, dried, and stained with 0.1% crystal violet which was again rinsed well with deionized water leaving the stained biofilm at the bottoms of the wells. This biofilm was dissolved in 200 μL of 30% acetic acid and absorbance at 562 nm was read to determine amount of biofilm formation. Each assay was carried out at least in triplicate. Biofilm inhibitor concentration (IC₅₀) of the compounds was determined by serial dilutions from 0 μM-50 μM (1% DMSO).

S. mutans, S. Gordonii, and S. sanguinis Growth Assays.

Effects of PEHA5, PEHA6, HA5 or HA6 on S. mutans and commensal bacterial growth were evaluated using the growth assay as described^{[62]}. S. mutans UA159, S. gordonii DL1, S. sanguinis SK36, cultures were grown for 24 h under 5% CO₂ at 37° C. These cultures were then reinoculated with fresh THB (1:5) until OD₄₇₀=1 when the bacteria were ready to be used. Different concentrations of the PEHA5, PEHA6, HA5 or HA6 were assayed in CDM with 1% of the bacteria, 1% sucrose and 1% DMSO in 96 well plates. The 96 well plates were incubated under 5% CO₂ at 37° C. for 16 h. Growth of the bacteria was read after 16 h at OD₄₇₀. Each assay was carried out at least in triplicate.

### Gtf Inhibition is Determined by Glucan Quantification Assays.

Gtf inhibition assays were performed to assess the ability of HA6 to inhibit the Gtfs and glucan production using a reported procedure and IC₅₀ value was calculated^{[44, 45]} Overnight cultures of S. mutans UA159 were centrifuged (6500 rpm, 4° C., 10 min) to remove the cells. Supernatant was mixed with ethanol (1:1) and incubated at −80° C. for 1 h. The precipitated Gtfs were palleted using centrifugation and resuspended in CDM (1 mL). 10 μL of Gtfs suspension in CDM was assayed on Ibidi slides with varying concentrations of PEHA5 or HA6, 1% sucrose, 1% DMSO and 1 μM Cascade blue dextran conjugated dye in CDM. The slides were then incubated at 37° C. with 5% CO₂ for 16 h after which, the wells of Ibidi slides were gently rinsed with 1×PBS and imaged using fluorescence microscopy. The images obtained were processed in ImageJ to quantify glucans and graphed in GraphPad Prism 10.0.2.

Fabrication of pH-Responsive Polymer Vesicles

Materials. Hydroxyl terminated polydimethylsiloxane (PDMS, average Mₙ~5600, Sigma-Aldrich) was dried overnight in vacuum at 40° C. Potassium ethyl xanthogenate (96%, Sigma-Aldrich) and 2,2′-azobis(2-methylpropionitrile (98%, Sigma-Aldrich) were recrystallized before synthesis from methanol and acetone, respectively, and dried in vacuum at 20° C. Acetonitrile (certified ACS grade), tetrahydrofuran (THF, HPLC grade), and 1-vinyl-2-pyrrolidone (VPON, 99%) were purchased from Fisher Scientific and distilled before use. Diethyl ether (anhydrous), methanol, sodium hydroxide, hydrochloric acid, anhydrous sodium sulfate, and pyridine were purchased from Fisher Scientific and used as received. 2-Bromo-2-propionyl bromide (98%, TCI) was stored under protective argon (Airgas) atmosphere and used as received.

Synthesis of PVPON₈-b-PDMS₆₅-b-PVPON; Triblock Copolymer.

First, PDMS terminated with dihydroxyl groups (10.0 g, 1.8 mmol) and pyridine (2.9 mL, 38 mmol) were mixed in a 250-mL round-bottom flask with 100 ml of anhydrous diethyl ester in an ice bath. A solution of 2-bromopropionyl bromide (3.0 mL, 24 mmol) in anhydrous diethyl ester (20 mL) was added dropwise to the mixture over 1 h. The solution was then allowed to warm to room temperature and was stirred for 24 h. The precipitate was separated by filtration and washed with 1.0 M HCl solution (3 times, 50 mL), 1.0 M NaOH solution (3 times, 50 mL), deionized (DI) water (4 times, 100 mL) and then dried with anhydrous sodium sulfate. Then, polymer solution was concentrated in a rotary evaporator and dried overnight under vacuum at room temperature. Next, dried polymer (9.8 g, 1.7 mmol) was added to acetonitrile (200 mL) in a 500-mL round-bottom flask and mixed with pyridine (2.9 mL, 38 mmol). Potassium ethyl xanthogenate (1.09 g, 6.8 mmol) dispersion in 20 mL of acetonitrile was added dropwise. The mixture was then stirred at room temperature overnight. After the precipitate was collected by filtration, the crude product was dissolved in 200 mL of diethyl ether. The organic solution was washed sufficiently with 1.0 M HCl solution (3 times, 50 mL), 1.0 M NaOH solution (3 times, 50 mL), and DI water (4 times, 100 mL) and then dried with anhydrous Na₂SO₄. Then, polymer solution was concentrated in a rotary evaporator and dried overnight under vacuum at room temperature. The final PDMS-based macro-CTA was collected in 8.9 g and Mn was measured to be 7,070 Da based on the 1H NMR calculation of repeating units. The final PVPON₈-b-PDMS₆₄-b-PVPON₈ triblock copolymer was obtained via CTA-mediated polymerization of VPON. Macro-CTA (1.0 g, 0.19 mmol), VPON (2.0 g, 18 mmol), AIBN (33 mg, 0.21 mmol), and freshly distilled tetrahydrofuran (4 mL) were added in one 25 mL Schlenk flask (reactor) equipped with a magnetic stirring bar. The mixed solution was degassed by 3 freeze-pump-thaw cycles. The polymerization was initiated by immersion of the reaction mixture to the preheated oil bath at 65° C. After 2 h, the reaction was immediately quenched in a liquid N₂. Then, the reaction mixture was diluted twice with methanol, transferred to dialysis tubes (MWCO=500-1000 Da, Spectrum Laboratories), and dialyzed for 2 days in methanol. Then, polymer solution was concentrated in a rotary evaporator and dried overnight under vacuum at room temperature. ¹H NMR spectra of the copolymer (15 mg mL⁻¹ in CDCl₃) were collected on a Bruker 400 MHz NMR spectrometer.

Encapsulation of HA5 into Polymer Vesicles

HA5 loaded polymersomes were prepared using a nanoprecipitation method. For that, 1.0 mL of the PVPON₈-b-PDMS₆₄-b-PVPON; triblock-copolymer solution in ethanol (5.0 mg/mL) was added dropwise to 4.0 mL of the 2.5 mg HA5 (or HA6) solution in DI water at room temperature and left under stirring for 2 h. Then, the obtained solution was dialysed in DI water for 48 h using a Float-a-Lyzer (MWCO 1000 Da, Fisher Scientific) to remove ethanol, followed by dialysis in DI water for 72 h using a Float-a-Lyzer (MWCO 100 kDa, Fisher Scientific) to remove an excess of the drug. The hydrodynamic size of empty and encapsulated polymersomes was measured using a Nano-ZS Zetasizer (Malvern Pananalytical) equipped with a He—Ne laser (663 nm) at 25° C. Drug concentration was calculated using a NanoDrop One Microvolume UV-Vis spectrophotometer (Thermo Fisher).

### Impact of HA5 and HA6 on the Rat Oral Microbiome

We used Fischer 344 rats that were bred and maintained in trexler isolators for this experiment. Male and female rat pups were removed from isolators at 20 days of age and randomly assigned into treatment groups of 5 rats/group in cages with filter tops. Rats were then infected with S. mutans UA159 strain by oral swabbing daily for four consecutive days with a fresh overnight culture of S. mutans UA159. Rats were provided with caries promoting Teklad Diet 305 containing 5% sucrose (Harlan Laboratories, Inc., Indianapolis, IN) and sterile drinking water ad libitum. Oral swabs were taken 5 days post-infection and plated on Todd Hewitt (TH) agar plates and incubated at 37° C. in an environment of 5% CO₂ in the air to confirm colonization. Rats were weighed at weaning and then weekly throughout the experiment. One-week post-infection, the molars of the rats were brushed twice daily for 4 weeks with the test compounds using camel-hair brushes. Four treatment groups used in this study were: 1) HA5 (100 μM); 2) HA6 (100 μM); 3) NaF (250 ppm); and 4) infected untreated rats. Drinking water was withheld for 60 min following each treatment with the compound. Animals were weighed at weaning and at the termination of the experiment. On day 60, the rats were sacrificed using CO₂ followed by cervical dislocation or bilateral thoracotomy. The mandibles were surgically removed and cleaned of excess tissue to assess the level of bacteria present and the extent of caries formation. The right mandible from each rat was placed in a tube containing phosphate buffer (3 mL), placed on ice and sonicated (10 sec) to release bacteria from the molars. Each sample was serially diluted, plated on blood (BAP) and mitis-salivarius (MS) agar plates and incubated in an environment of 5% CO₂ in air at 37° C. to quantify the level of total bacteria and S. mutans present in the plaque. The right and left mandibles from each rat were then placed in 95% ethanol for 24 h. The mandibles will be cleaned and stained overnight with murexide solution. After drying, the mandibles were sectioned and scored for caries activity using the Keyes method Caries scores were recorded for the buccal, sulcal and proximal molar surfaces individually so that differences among the surfaces can be distinguished. Statistical significance in the mean caries scores, colony-forming units (CFU)/mandible and body weights between groups of rats were determined by one-way analysis of variance (ANOVA) with the Tukey-Kramer multiple comparison test using the InStat program (GraphPad Prism 10.0.2.). When determining the statistical significance between the two groups, an unpaired t-test was applied. Differences between groups were considered significant at a P-value<0.05.

In addition to recording buccal, sulcal and proximal molar surface caries scores and colony-forming units (CFU)/mandible and body weights between groups of rats as described above, plaque samples from rats treated or untreated were collected and DNA was extracted from each sample and used for PCR amplification of ~430 bp amplicons of 16S ribosomal DNA hypervariable regions V3 and V4, Illumina adaptors, and molecular barcodes as described^{[66]}. Barcoded PCR samples were sequenced at UAB Microbiome Research Core. The Ribosomal Database Project classifier was used to assign a taxonomic classification to each read in the representative set and a phylogenetic tree will be constructed from the representative sequences. The relative abundance of each OTU was examined at phylum, class, order, family, genus, and species levels. Alpha and beta diversity analysis of the oral microbial community was performed using MicrobiomeAnalyst 2.0^{[55]}. The ASV table containing the raw counts was filtered to remove low abundance features that were less than 10% prevalence in samples, and data scaling was performed using Total sum scaling (TSS) prior to the diversity analysis. Alpha diversity indices generated were used in GraphPad Prism 10.0.2 to generate the bar plots. All experimental protocols were approved by the University of Alabama at Birmingham Institutional Animal Care and Use Committee. The methods were carried out in accordance with the relevant guidelines and regulations.

### References for Example 2

1. Marsh P D, Dental Plaque as a Microbial Biofilm. Caries Res., 2004. 38: p. 204-211.
2. Vos T, Allen, C., Arora, M., Barber, R. M., Bhutta, Z. A., Brown, A., Carter, A., Casey, D. C., Charlson, F. J., Chen, A. Z., Coggeshall, M., Cornaby, L., Dandona, L., Dicker, D. J., Dilegge, T., Erskine, H. E., Ferrari, A. J., Fitzmaurice, C., Fleming, T., . . . . Murray, C. J. L., Global, Regional, and National Incidence, Prevalence, and Years Lived with Disability for 310 Diseases and Injuries, 1990-2015: A Systematic Analysis for the Global Burden of Disease Study 2015. Lancet, 2016. 388: p. 1545-1602.
3. Hamada S and Slade H D, Biology, Immunology, and Cariogenicity of Streptococcus mutans. Microbiol. Rev., 1980. 44: p. 331-384.
4. Loesche W J, Role of Streptococcus mutans in Human Dental Decay. Microbiol. Rev., 1986. 50: p. 353-380.
5. Jenkinson H F and Lamont R J, Oral Microbial Communities in Sickness and in Health. Trends Microbiol., 2005. 13: p. 589-595.
6. Kolenbrander P E, Andersen R N, Blehert D S, Egland P G, Foster J S, and Palmer R J, Jr., Communication Among Oral Bacteria. Microbiol. Mol. Biol. Rev., 2002. 66: p. 486-505.
7. Kuramitsu H K, He X, Lux R, Anderson M H, and Shi W, Interspecies Interactions Within Oral Microbial Communities. Microbiol. Mol. Biol. Rev., 2007. 71: p. 653-670.
8. Kouidhi B, Al Qurashi Y M, and Chaieb K, Drug resistance of bacterial dental biofilm and the potential use of natural compounds as alternative for prevention and treatment. Microb Pathog, 2015. 80: p. 39-49.
9. Roberts A P and Mullany P, Oral biofilms: a reservoir of transferable, bacterial, antimicrobial resistance. Expert Rev Anti Infect Ther, 2010. 8: p. 1441-1450.
10. Saini R, Saini S, and Sharma S, Biofilm: A dental microbial infection. Journal of Natural Science, Biology, and Medicine, 2011. 2: p. 71-75.
11. Bowden G H and Hamilton I R, Survival of Oral Bacteria. Crit. Rev. Oral Biol. Med., 1998. 9: p. 54-85.
12. Quivey R G, Jr., Kuhnert W L, and Hahn K, Adaptation of Oral Streptococci to Low pH. Adv. Microb. Physiol., 2000. 42: p. 239-274.
13. Yamashita Y, Bowen W H, Burne R A, and Kuramitsu H K, Role of the Streptococcus mutans gtf Genes in Caries Induction in the Specific Pathogen-free Rat Model. Infect. Immun., 1993. 61: p. 3811-3817.
14. Aoki H, Shiroza T, Hayakawa M, Sato S, and Kuramitsu H K, Cloning of a Streptococcus mutans Glucosyltransferase Gene Coding for Insoluble Glucan Synthesis. Infect. Immun., 1986. 53: p. 587-594.
15. Hanada N and Kuramitsu H K, Isolation and Characterization of the Streptococcus mutans gtfC Gene Coding for Synthesis of Both Soluble and Insoluble Glucans. Infect. Immun., 1988. 56: p. 1999-2005.
16. Hanada N and Kuramitsu H K, Isolation and Characterization of the Streptococcus mutans gtfD Gene Coding for Primer-dependent Soluble Glucan Synthesis. Infect. Immun., 1989. 57: p. 2079-2085.
17. Monchois V, Willemot R M, and Monsan P, Glucansucrases: Mechanism of Action and Structure-function Relationships. FEMS Microbiol. Rev., 1999. 23: p. 131-151.
18. Ahirwar P, Kozlovskaya V, Nijampatnam B, Rojas E M, Pukkanasut P, Inman D, Dolmat M, Law A C, Schormann N, Deivanayagam C, Harber G J, Michalek S M, Wu H, Kharlampieva E, and Velu S E, Hydrogel-Encapsulated Biofilm Inhibitors Abrogate the Cariogenic Activity of Streptococcus mutans. J. Med. Chem., 2023. 66: p. 7909-7925.
19. Aframian D J, Davidowitz T, and Benoliel R, The Distribution of Oral Mucosal pH Values in Healthy Saliva Secretors. Oral Dis., 2006. 12: p. 420-423.
20. Baliga S, Muglikar S, and Kale R, Salivary pH: A Diagnostic Biomarker. J. Indian Soc. Periodont., 2013. 17: p. 461-465.
21. Bardow A, Pederson AML, and Nauntofte B, Saliva. In: Miles T S, Nauntofte B, Svensson P eds. Clin. Oral Physiol., 2004: p. 17-33.
22. Lazarchik D A and Filler S J, Effects of Gastroesophageal Reflux on the Oral Cavity. Am. J. Med., 1997. 103: p. 107S-113S.
23. Markitziu A and Aframian D, Gastro-Intestinal Disorders. Br. Dent. J., 1997. 182: p. 207.
24 Robb N D, Smith B G, and Geidrys-Leeper E, The Distribution of Erosion in the Dentitions of Patients with Eating Disorders. Br. Dent. J., 1995. 178: p. 171-175.
25. Balhaddad A A, Kansara A A, Hidan D, Weir M D, Xu HHK, and Melo M A S, Toward dental caries: Exploring nanoparticle-based platforms and calcium phosphate compounds for dental restorative materials. Bioactive Materials, 2019. 4: p. 43-55.
26. Carrouel F, Viennot S, Ottolenghi L, Gaillard C, and Bourgeois D, Nanoparticles as Anti-Microbial, Anti-Inflammatory, and Remineralizing Agents in Oral Care Cosmetics: A Review of the Current Situation. Nanomaterials, 2020. 10: p. 140.
27. Chen H, Gu L, Liao B, Zhou X, Cheng L, and Ren B, Advances of Anti-Caries Nanomaterials. Molecules, 2020. 25: p. 5047.
28. Song W and Ge S, Application of Antimicrobial Nanoparticles in Dentistry. Molecules 2019. 24: p. 1033.
29. Zhao Z, Ding C, Wang Y, Tan H, and Li J, pH-Responsive polymeric nanocarriers for efficient killing of cariogenic bacteria in biofilms. Biomater Sci, 2019. 7: p. 1643-1651.
30. Sims K R, Maceren J P, Liu Y, Rocha G R, Koo H, and Benoit DSW, Dual Antibacterial Drug-loaded Nanoparticles Synergistically Improve Treatment of Streptococcus mutans Biofilms. Acta Biomater., 2020. 115: p. 418-431.
31. Yi Y, Wang L, Chen L, Lin Y, Luo Z, Chen Z, Li T, Wu J, and Zhong Z, Farnesal-loaded pH-Sensitive Polymeric Micelles Provided Effective Prevention and Treatment on Dental Caries. J. Nanobiotech., 2020. 18: p. 89.
32. Seneviratne C J, Leung K C—F, Wong C—H, Lee S—F, Li X, Leung P C, Lau C B S, Wat E, and Jin L, Nanoparticle-Encapsulated Chlorhexidine against Oral Bacterial Biofilms. Plos One, 2014. 9: p. e103234.
33. Zhang J F, Wu R, Fan Y, Liao S, Wang Y, Wen Z T, and Xu X, Antibacterial Dental Composites with Chlorhexidine and Mesoporous Silica. J. Dent. Res., 2014. 93: p. 1283-1289.
34. Nguyen S, Hiorth M, Rykke M, and Smistad G, Polymer Coated Liposomes for Dental Drug Delivery: Interactions with Parotid Saliva and Dental Enamel. Eur. J. Pharm. Sci., 2013. 50: p. 78-85.
35. Feitosa S A, Palasuk J, Kamocki K, Geraldeli S, Gregory R L, Platt J A, Windsor L J, and Bottino M C, Doxycycline-Encapsulated Nanotube-Modified Dentin Adhesives. J. Dent. Res., 2014. 93: p. 1270-1276.
36. Zhou Y, Yang J, Lin Z, Li J, Liang K, Yuan H, Li S, and Li J, Triclosan-Loaded Poly(amido amine) Dendrimer for Simultaneous Treatment and Remineralization of Human Dentine. Colloids Surf. B., 2014. 115: p. 237-243.
37. Ferji K, Nouvel C, Babin J, Li M H, Gaillard C, Nicol E, Chassenieux C, and Six J L, Polymersomes from Amphiphilic Glycopolymers Containing Polymeric Liquid Crystal Grafts. ACS Macro. Lett., 2015. 4: p. 1119-1122.
38. Ghorbanizamani F, Moulahoum H, Zihnioglu F, and Timur S, Nanohybrid carriers: the yin-yang equilibrium between natural and synthetic in biomedicine. Biomat. Sci., 2020. 8: p. 3237-3247.
39. Battaglia G and Ryan A J, Bilayers and interdigitation in block copolymer vesicles. J. Am. Chem. Soc., 2005. 127: p. 8757-8764.
40. Agut W, Brulet A, Schatz C, Taton D, and Lecommandoux S, pH and temperature responsive polymeric micelles and polymersomes by self-assembly of poly[2-(dimethylamino)ethyl methacrylate]-b-poly(glutamic acid) double hydrophilic block copolymers. Langmuir, 2010. 26: p. 10546-10554.
41. Kita-Tokarczyk K, Grumelard J, Haefele T, and Meier W, Block copolymer vesicles—using concepts from polymer chemistry to mimic biomembranes. Polymer, 2005. 46: p. 3540-3563.
42. Liang X, Liu F, Kozlovskaya V, Palchak Z, and Kharlampieva E, Thermoresponsive Micelles from Double LCST-Poly(3-methyl-N-vinylcaprolactam) Block Copolymers for Cancer Therapy. ACS Macro Letters, 2015. 4: p. 308-311.
43. Liu F, Kozlovskaya V, Zavgorodnya O, Martinez-Lopez C, Catledge S, and Kharlampieva E, Encapsulation of anticancer drug by hydrogen-bonded multilayers of tannic acid. Soft Matter, 2014. 10: p. 9237-9247.
44. Huffines J T and Scoffield J A, Disruption of Streptococcus mutans and Candida albicans Synergy by a Commensal Streptococcus. Sci. Rep., 2020. 10: p. 19661.
45. Huffines J T, Stoner S N, Baty J J, and Scoffield J A, Nitrite Triggers Reprogramming of the Oral Polymicrobial Metabolome by a Commensal Streptococcus. Front. Cell Infect. Microbiol., 2022. 12: p. 833339.
46. Rarey M, Kramer B, Lengauer T, and Klebe G, A Fast Flexible Docking Method using an Incremental Construction Algorithm. J. Mol. Biol., 1996. 261: p. 470-489.
47. BioSolveIT GmbH, SeeSAR version 13.0.1. 2023: Sankt Augustin, Germany.
48. Warren G L, Andrews C W, Capelli A-M, Clarke B, LaLonde J, Lambert M H, Lindvall M, Nevins N, Semus S F, Senger S, Tedesco G, Wall I D, Woolven J M, Peishoff C E, and Head M S, A Critical Assessment of Docking Programs and Scoring Functions. J. Med. Chem., 2006. 49: p. 5912-5931.
49. Zhang Q, Ma Q, Wang Y, Wu H, and Zou J, Molecular mechanisms of inhibiting glucosyltransferases for biofilm formation in Streptococcus mutans. Int. J. Oral. Sci., 2021. 13: p. 30.
50. Banas J A, Lynch D J, Michalek S M, Zhu M, Drake D, and Qian F, Cariogenicity of Streptococcus mutans Glucan-Binding Protein Deletion Mutants. Oral Health Dent. Manag., 2013. 12: p. 191-199.
51. Hazlett K R, Michalek S M, and Banas J A, Inactivation of the gbpA Gene of Streptococcus mutans Increases virulence and Promotes In Vivo Accumulation of Recombinations Between the Glucosyltransferase B and C Genes. Infect. Immun., 1998. 66: p. 2180-2185.
52. Michalek S M, McGhee J R, and Navia J M, Virulence of Streptococcus mutans: a sensitive method for evaluating cariogenicity in young gnotobiotic rats. Infect. Immun., 1975. 12: p. 69-75.
53. Michalek S M, McGhee J R, Shiota T, and Devenyns D, Virulence of Streptococcus mutans: Cariogenicity of S. mutans in Adult Gnotobiotic Rats. Infect. Immun., 1977. 15: p. 466-471.
54. Palmer S R, Crowley P J, Oli M W, Ruelf M A, Michalek S M, and Brady L J, YidC1 and YidC2 are functionally distinct proteins involved in protein secretion, biofilm formation and
cariogenicity of Streptococcus mutans. Microbiology, 2012. 158: p. 1702-1712.
55. Lu Y, Zhou G, Ewald J, Pang Z, Shiri T, and Xia J, MicrobiomeAnalyst 2.0: comprehensive statistical, functional and integrative analysis of microbiome data. Nucleic Acids Res., 2023. 51: p. W310-W318.
56. Guo L, McLean J S, Yang Y, Eckert R, Kaplan C W, Kyme P, Sheikh O, Varnum B, Lux R, Shi W, and He X, Precision-guided antimicrobial peptide as a targeted modulator of human microbial ecology. Proc. Natl. Acad. Sci. USA, 2015. 112: p. 7569-7574.
57. Garcia S S, Blackledge M S, Michalek S, Su L, Ptacek T, Eipers P, Morrow C, Lefkowitz E J, Melander C, and Wu H, Targeting of Streptococcus mutans Biofilms by a Novel Small Molecule Prevents Dental Caries and Preserves the Oral Microbiome. J. Dent. Res., 2017. 96: p. 807-814.
58. Yang Y, Kozlovskaya V, Dolmat M, Song Y, Qian S, Urban V S, Cropek D, and Kharlampieva E, Temperature controlled transformations of giant unilamellar vesicles of amphiphilic triblock copolymers synthesized via microfluidic mixing. Appl. Surf. Sci. Adv., 2021. 5: p. 100101.
59. Yang Y, Kozlovskaya V, Zhang Z, Xing C, Zaharias S, Dolmat M, Qian S, Zhang J, Warram J M, Yang E S, and Kharlampieva E, Poly(N-vinylpyrrolidone)-block-Poly(dimethylsiloxane)-block-Poly(N-vinylpyrrolidone) Triblock Copolymer Polymersomes for Delivery of PARP1 siRNA to Breast Cancers. ACS Appl. Bio Mater., 2022. 5: p. 1670-1682.
60. Liu F, Kozlovskaya V, Medipelli S, Xue B, Ahmad F, Saeed M, Cropek D, and Kharlampieva E, Temperature-Sensitive Polymersomes for Controlled Delivery of Anticancer Drugs. Chemistry of Materials, 2015. 27: p. 7945-7956.
61. Loo C, Corliss D, and Ganeshkumar N, Streptococcus gordonii biofilm formation: identification of genes that code for biofilm phenotypes. J Bacteriol, 2000. 182: p. 1374-1382.
62. Liu C, Worthington R J, Melander C, and Wu H, A New Small Molecule Specifically
Inhibits the Cariogenic Bacterium Streptococcus mutans in Multispecies Biofilms. Antimicrob. Agents Chemother., 2011. 55: p. 2679-2687.
63. Zhang Q, Nguyen T, McMichael M, Velu S E, Zou J, Zhou X, and Wu H, New Small-Molecule Inhibitors of Dihydrofolate Reductase Inhibit Streptococcus mutans. Int. J. Antimicrob. Agents, 2015. 46: p. 174-182.
64. Schrödinger L, The PyMOL Molecular Graphics System, Version 2.4.2.
65. Keyes P H, Dental Caries in the Molar Teeth of Rats. II. A Method for Diagnosing and Scoring Several Types of Lesions Simultaneously. J. Dent. Res., 1958. 37: p. 1088-1099.
66. Bruce-Keller A J, Salbaum J M, Luo M, Blanchard Et, Taylor C M, Welsh D A, and Berthoud H R, Obese-type gut microbiota induce neurobehavioral changes in the absence of obesity. Biol. Psychiatry, 2015. 77: p. 607-615.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

1. A composition comprising a plurality of hydrogel particles and a plurality of small molecules, wherein the small molecules are encapsulated inside the hydrogel particles and wherein the small molecules have the following structure: 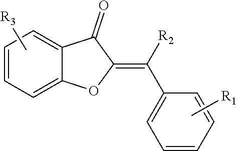
wherein each R₁ is independently selected from hydrogen, a substituted or unsubstituted C1-C3 alkyl group, a substituted or unsubstituted C1-C3 alkoxyl group, and OH, wherein R₂ is hydrogen, a substituted or unsubstituted C1-C3 alkyl group, a substituted or unsubstituted C1-C3 alkoxyl group, and OH, wherein each R₃ is independently selected from hydrogen, a substituted or unsubstituted C1-C3 alkyl group, a substituted or unsubstituted C1-C3 alkoxyl group, and OH.

2. The composition of claim 1, wherein each R₁ is independently selected from hydrogen, methoxy, and OH.

3. The composition of claim 1, wherein the structure includes 2 R₁ groups and the each R₁ group is methoxy or OH.

4. The composition of claim 1, wherein the structure includes 3 R₁ groups and the each R₁ group is methoxy or OH.

5. The composition of claim 1, wherein the structure is 2-[(2′,4′,5′-trihydroxyphenyl)methylidene]-2,3-dihydro-1-benzofuran-3-one.

6. The composition of claim 1, wherein the structure is 2-[(3′,4′,5′-trihydroxyphenyl)methylidene]-2,3-dihydro-1-benzofuran-3-one.

7. The composition of claim 1, wherein the hydrogel particle is made of a polymer or co-polymers of poly(N-vinylpyrrolidone) (PVPON) and poly(dimethylsiloxane) (PDMS).

8. The composition of claim 1, wherein the hydrogel particles is a vesical comprising the small molecules within the vesicle.

9. (canceled)

10. The composition of claim 1, wherein the hydrogel particles is made of cross-linked poly(methacrylic acid) (PMAA), wherein the small molecules are within the PMAA.

11. The composition of claim 8, wherein the hydrogel particles are each comprised of 3 to 7 layers of cross-linked poly(methacrylic acid) (PMAA), wherein the small molecules are within the layer of the PMAA.

12. The composition of claim 11, wherein the hydrogel particles have a cubic shape.

13. The composition of claim 11, wherein the hydrogel particles have a diameter of about 1 μm to about 10 μm.

14. The composition of claim 11, wherein the hydrogel particles further comprise pH-sensitive cross-linkers.

15. The composition of claim 12, wherein the pH-sensitive cross-linkers are selected from the group consisting of ethylene diamine, cystamine, dithiobis(succinimidyl propionate), adipic acid dihydrazide, or any combination thereof.

16. The composition of claim 1, wherein the loading capacity of the hydrogel particles is about 0.003 to about 0.008 ng of the small molecule per particle.

17. A pharmaceutical composition comprising a therapeutically effective amount of the composition of claim 1 to treat a condition.

18. The pharmaceutical composition of claim 17, wherein the pharmaceutical composition further comprises a pharmaceutically acceptable carrier.

19. The pharmaceutical composition of claim 18, wherein the pharmaceutically acceptable carrier is a dentifrice.

20. The pharmaceutical composition of claim 17, wherein condition is dental caries.

21. A method for treating dental caries comprising contacting a biofilm or contacting a cell capable of forming a biofilm with a therapeutically effective amount of the composition or pharmaceutical composition of claim 1.

22. The method of claim 21, where the cell capable of forming a biofilm is Streptococcus mutans.
